(19) United States
(12) Patent Application Publication
(10) Pub. No.: US 2009/0074845 A1
(43) Pub. Date: Mar. 19, 2009

(54) TRANSDERMAL ACTIVE PRINCIPLE DELIVERY MEANS

(75) Inventors: Ian Stuart Pardoe, West Midlands (GB); Christopher Hartley, West Midland (GB)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(73) Assignee: HENDERSON MORLEY PLC, Birmingham, West Midlands (GB)

(21) Appl. No.: 12/065,591

(22) PCT Filed: Aug. 22, 2006
(86) PCT No.: PCT/GB2006/003143

§ 371 Date: Apr. 16, 2008

(30) Foreign Application Priority Data
Sep. 2, 2005 (GB) 0517838.9

(51) Int. Cl. A61K 9/70; A61K 31/34; A61K 31/192; A61K 31/70

(52) U.S. Cl. 424/449; 514/471; 514/571; 514/26

(57) ABSTRACT

A transdermal active principle delivery means comprises a skin adherent or otherwise skin-tolerant substrate applicable to a skin area affected by DNA virus, which substrate includes a composition for treating DNA comprising a transdermally effective carrier medium including at least one active principle selected from the group consisting of diuretic agents and/or cardiac glycoside agents.

The present invention is concerned with transdermal delivery means comprising active principles for use in anti-viral treatments and in particular, to such delivery means useful in the prophylactic and therapeutic treatment of DNA viral infections such as Herpes virus infections, and in particular, for the treatment of HPV (human papillomavirus) infections as typically cause unsightly and uncomfortable warts.

Herpes viruses are DNA viruses, having a central core of DNA within a proteinaceous structure. DNA carries the genetic code to reproduce the virus. Viruses must infect living ‘host’ cells to reproduce. There are numerous well characterised viral proteins including important enzymes which act as ideal targets for antiviral chemotherapy. These include DNA polymerase and thymidine kinase essential for DNA replication. The replication of viral DNA is essential for virus infectivity. It is known replication of infecting viruses can alter the natural ionic balances within the living host cells.

EP-A-0442744 discloses the use of certain glycosides to treat Herpes Simplex Virus and Varicella Zoster Virus. WO 00/10574 discloses the use of a loop diuretic in the treatment of a retrovirus, in this case, to treat HIV infection. We have now surprisingly found that transdermal application of a loop diuretic and/or cardiac glycoside across the skin barrier is feasible and can be effective in the therapeutic treatment of DNA viral infections and especially in the topical treatment of skin areas showing symptoms of Papilloma virus infection such as warts.

According to the invention in one aspect there is provided transdermal active principle delivery means comprising a skin adherent or otherwise skin-tolerant substrate applicable to a skin area affected by DNA virus, which substrate includes a composition for treating DNA virus infestation within a transdermally effective carrier medium of at least one active principle selected from the group consisting of loop diuretic agents and/or cardiac glycoside agents.

In another aspect of the invention provides making delivery means, comprising forming a composition comprising one or both of a loop diuretic and/or cardiac glycoside in a transdermally effective carrier medium and applying composition to a set or tacky Collodion layer.

The loop diuretic as indicated above may be selected from a wide range of available such agents. Preferably the loop diuretic is any one or more of furosemide, bumetanide, ethacrynic acid or torasemide. Most preferably the loop diuretic consists of furosemide. According to our studies but without wishing to be bound by any theoretical postulations, loop diuretics apparently mediate their antiviral effects through alteration to the cellular concentration of ions, cellular ionic balances, cellular ionic milieu and electrical potentials.

Furosemide is an anthrilic acid derivative, chemically 4-chloro-N-furfuryl-5-sulfamoylanthranilic acid. Practically insoluble in water at neutral pH, furosemide is freely soluble in alkali. Furosemide exerts its physiological effect by inhibition of the transport of chloride ions across cell members. Furosemide is a loop diuretic with a short duration of action. It is used for treating oedema due to hepatic, renal or cardiac failure and for treating hypertension. The bioavailability of furosemide ranges from about 60% to about 70% and is primarily excreted by filtration and secretion as unchanged drug. Furosemide acts on the Na+/K+/2Cl— co-transformer. For its diuretic effect, its predominant action is in the ascending limb of the loop of Henlé in the kidney, hence the generally accepted term ‘loop diuretic’. Loop diuretics markedly promote K⁺ excretion, leaving cells depleted in intracellular potassium. This may lead to the most significant complication of long term systemic furosemide usage namely a lowered serum potassium. Without wishing to be bound by any theoretical considerations, we postulate that cellular ionic potassium depletion makes loop diuretics useful against DNA viruses.

Evidence suggests that the major biotransformation product of furosemide is a glucoronide. Furosemide is extensively bound to plasma proteins, mainly albumin. Plasma concentrations ranging from 1 to 400 mcg/ml are 91-99% bound in healthy individuals. The unbound fraction ranges between 2.3-4.4% at therapeutic concentrations. The terminal half life of furosemide is approximately 2 hours and it is predominantly excreted in the urine.

The cardiac glycosides as indicated above may be any one or more of digoxin, digitoxin, medigoxin, lanatoside C, proscillaridin, k strophantin, peruvoside and ouabain. Most preferably digoxin is used alone. Plants of the digitalis species (e.g. digitalis purpura, digitalis lanata) contain cardiac glycosides such as digoxin and digitoxin which are known collectively as digitalis. Other plants contain cardiac glycosides which are chemically related to the digitalis glycosides and these are often also referred to as digitalis. Thus, the term digitalis is used to designate the whole group of glycosides; the glycosides are composed of two components, a sugar and a cardenolide. Ouabain is derived from an African plant Strophanthus gratus (also known a strophanthidin G) and is available in intravenous form (it is not absorbed orally) and is used for many laboratory experiments in the study of glycosides, because of its greater solubility. It has a virtually identical mode of action as digoxin.

Digoxin is described chemically as (3b,5b,12b)-3-[0-,6-didioxy-b-D-riob-hexapyranosyl-(1″4)-0-2,6-dideoxy-b-D-ribo-hexapyranosyl-(1″4)-2,6-dideoxy-b-D-ribo-hexapyranozyl)oxy]-12,14-dihydroxy-card-20-22)-enolide. Its molecular formula is C₄₁H₆₄0₁₄, and its molecular weight is 780.95. Digoxin exists as odourless white crystals that melt with decomposition above 230° C. The drug is practically insoluble in water and in ether; slightly soluble in diluted (50%) alcohol and in chloroform; and freely soluble in pyridine.

Because some patients may be particularly susceptible to side effects with digoxin, the dosage of the drug is selected and adjusted carefully as the clinical condition of the patient warrants.

At the cellular level digitalis exerts its main effect by the inhibition of the sodium transport enzyme sodium potassium adenosine triphosphatase (Na/K ATPase); this is directly responsible for the electrophysiological effects on heart muscle and according to theoretical postulations but without being bound thereby, also its activity against DNA viruses.

A particularly preferred combination in the compositions is the loop diuretic furosemide coupled with the cardiac glycoside digoxin. It is within the scope of the invention to provide separate delivery means for the sequential application of the two active principles, in use separated by a short time period.

Studies (including X-ray microanalysis) have demonstrated the anti-viral DNA effects of delivery means including compositions according to the invention are attributable to depletion of virus-infected host intracellular potassium ions. Briefly these studies demonstrate:
replacement of lowered potassium will restore DNA synthesis and hence viral replication;
use of furosemide and digoxin in combination have comparable effects to potassium depletion;
the level of potassium depletion is sufficient to allow normal cell function;
the potassium depletion has no cytotoxic effects.

Thus, by altering the cellular concentrations of ions, cellular ionic balances, cellular ionic milieu and cellular electrical potentials by the application of a loop diuretic and for a cardiac glycoside, cell metabolism can be altered without detriment to normal functions within the cell but so that DNA virus replication is inhibited. Accordingly, use of a loop diuretic and/or a cardiac glycoside within a transdermally effective carrier is of benefit in preventing or controlling virus replication by inhibiting the replication of viral DNA. Anti-viral efficacy has been demonstrated against the DNA viruses HSV1 and HSV2, CMV, VZV, Mammalian Herpes Viruses and papoviruses; adenoviruses.

We believe that efficacy will also be shown against parvoviruses; Pseudorabies; hepadnoviruses and poxviruses.

The transdermal delivery means of the invention may be conveniently adapted for external administration by adhesion to a site on the skin affected by DNA virus such as Herpes simplex virus. Topical applications effective transdermally across the skin barrier are likely to be most useful. The compositions within the delivery means may be for specially formulated for slow release. It is a much preferred feature of the invention that the compositions are formulated for topical transdermally effective application. Other ingredients within the compositions may be present, provided that they do not compromise the anti-viral activity; examples include preservatives, adjuncts, excipients, thickeners and solvents. Preferably the invention provides delivery means including a combination of furosemide and digoxin as a topical application in a buffered saline formulation for the treatment of corneal eye infections.

A preferred application of this invention is the use of local concentrations of loop diuretic and cardiac glycoside for the highly effective treatment of HPV virus infections causing warts.

The invention will now be described by way of illustration only with reference to the following examples.

Examples 1 to 3 are included by way of illustration to show the effects including synergistic effects of compositions comprising digoxin and furosemide against cells infected with HSV virus. It should be emphasised here that such examples are not however demonstrating transdermally effective delivery means entirely within the scope of the invention, but are nonetheless useful indicators of efficacy.

### EXAMPLE 1

Bioassays with herpes simplex virus in vitro were undertaken to follow the anti-viral activity of the simultaneous administration of furosemide (1 mg/ml) and digoxin (30 mcg/ml). Culture and assay methods follow those described by Lennette and Schmidt (1979) for herpes simplex virus and Vero cells with minor modifications.

### Herpes Simplex Strains Used:

Type 1 herpes simplex strain HFEM is a derivative of the Rockerfeller strain HF (Wildy 1955), and Type 2 herpes simplex strain 3345, a penile isolate (Skinner et a! 1977) were used as prototype strains. These prototypes were stored at −80° C. until needed.

### Cell Cultures:

African Green Monkey kidney cells (vero) were obtained from the National Institute of Biological Standards and Control UK and were used as the only cell line for all experiments in the examples.

### Culture Media:

Cells and viruses were maintained on Glasgows modified medium supplemented with 10% foetal bovine serum.

### Results:

Inhibition of hsvl

| Multiplicity of | | | Effect of |
|---|---|---|---|
| infection (dose | | | furosemide |
| of | Effect of furosemide | Effect of digoxin | and digoxin in |
| virus) | alone | alone | combination |
| High | | | +++ |
| Medium | + | + | ++++ |
| Low | + | ++ | ++++ |

This example demonstrates that virus activity was almost eliminated by applying low concentrations of the stock furosemide and glycoside solution to Vero cells infected with HSVI. At higher concentrations virus activity was completely prevented. The anti-viral effects of this stock solution were far greater than the effects of furosemide or digoxin alone. There was no direct virucidal activity on extracellular virus.

These experiments were repeated using a HSV2 strain, and almost identical results were obtained.

### EXAMPLE 2

The method of Example 1 was repeated using type 1 herpes virus strain kos. Similar results were obtained.

### EXAMPLE 3

In vitro bioassays were undertaken to follow the anti-viral activity of furosemide and digoxin when applied both simultaneously and alone.

The compositions were applied to different types of vero cells (African green monkey kidney cells and BHK1 cells) and infected with type 2 herpes simplex virus (strains 3345 and 180) at low, intermediate, and high multiplicities of infection (MOI). Inhibition of virus replication was scored on the scale:

| | |
|---|---|
| no inhibition | − |
| 20% inhibition | + |
| 40% inhibition | ++ |
| 60% inhibition | +++ |
| 80% inhibition | ++++ |
| 100% inhibition | +++++ |

T denotes drug toxicity.

The following results were obtained using African green monkey kidney cells and type 2 herpes simplex strain 3345:

| | Furosemide 0 mg/ml | Furosemide 0.5 mg/ml | Furosemide 1.0 mg/ml | Furosemide 2 mg/ml |
|---|---|---|---|---|
| LOW MOI HSV2 | | | | |
| Digoxin 0 mcg/ml | − | + | +++ | T |
| Digoxin 15 mcg/ml | − | + | +++ | T |
| Digoxin 30 mcg/ml | +++ | +++ | +++++ | T |
| Digoxin 45 mcg/ml | T | T | T | T |
| INT. MO! HSV2 | | | | |
| Digoxin 0 mcg/ml | − | + | +++ | T |
| Digoxin 15 mcg/ml | − | + | +++ | T |
| Digoxin 30 mcg/ml | | ++ | +++++ | T |
| Digoxin 45 mcg/ml | T | T | T | T |
| HIGH MOI HSV2 | | | | |
| Digoxin 0 mcg/ml | − | − | ++ | T |
| Digoxin 15 mcg/ml | − | − | +++ | T |
| Digoxin 30 mcg/ml | − | − | +++++ | T |
| Digoxin 45 mcg/ml | T | T | T | T |

The greatest effect of digoxin alone (+++) occurred on application of 30 mcg/ml digoxin at low multiplicity of infection only.

The greatest effect of furosemide alone (+++) occurred on application of 1 mg/ml furosemide at low and intermediate multiplicities of infection.

When the loop diuretic and cardiac glycoside were simultaneously applied to the infected cells, the greatest effect (+++++) was achieved using dioxin at 30 mcg/ml and furosemide at 1 mg/ml. 100% inhibition of HSV2 replication was shown at low, intermediate and high multiplicities of infection.

Similar results were obtained using other combinations of vero cells and type 2 herpes simplex strains.

This example demonstrates that replication of HSV2 is not maximally inhibited by applying furosemide or digoxin alone. However, in combination furosemide and digoxin completely inhibited HSV2 replication.

### EXAMPLE 4

This example demonstrates the in vitro release and permeation of digoxin and furosemide from transdermal delivery devices. Delivery systems were evaluated as formulations for this application in the presence and absence of additional excipients to aid both release and penetration. Three acrylic polymer-based glues were utilised.

### Materials

Digoxin and furosemide were purchased from Sigma, UK. Durotak acrylic glues were sourced from National Starch and Chemical Company. Duro-tak 87-900A (Glue 1), 87-2052 (Glue 2) and 87-201A (Glue 3) were used. All solvents and chemicals used for the release and permeability were purchased from Sigma. The silicone sheeting that was used as a synthetic skin barrier was purchased from Advanced Biotechnologies, USA.

### Methods

Formulation and in vitro evaluation of a transdermal patch for the delivery of digoxin and furosemide is outlined below.

### Development of an HPLC Method for Digoxin and Furosemide

For effective therapy drug must initially be released from a formulation prior to penetration of the skin; in each case the amount of drug release or the rate of penetration will need to be quantified. GHPLC offers a reliable means of quantifying the amount of drug that has been released. There are several published methods that detail HPLC analysis of both drugs. The HPLC used was Agilent Series 1100 with a Phenomenex C18 (150×4.60 mm 5 micro) column. The mobile phase was water, methanol and acetonitrile (40:30:30) and flowed at 1 ml/min. 20 μl of sample was injected and detected at 220 nm with a variable wavelength detector (VWD).

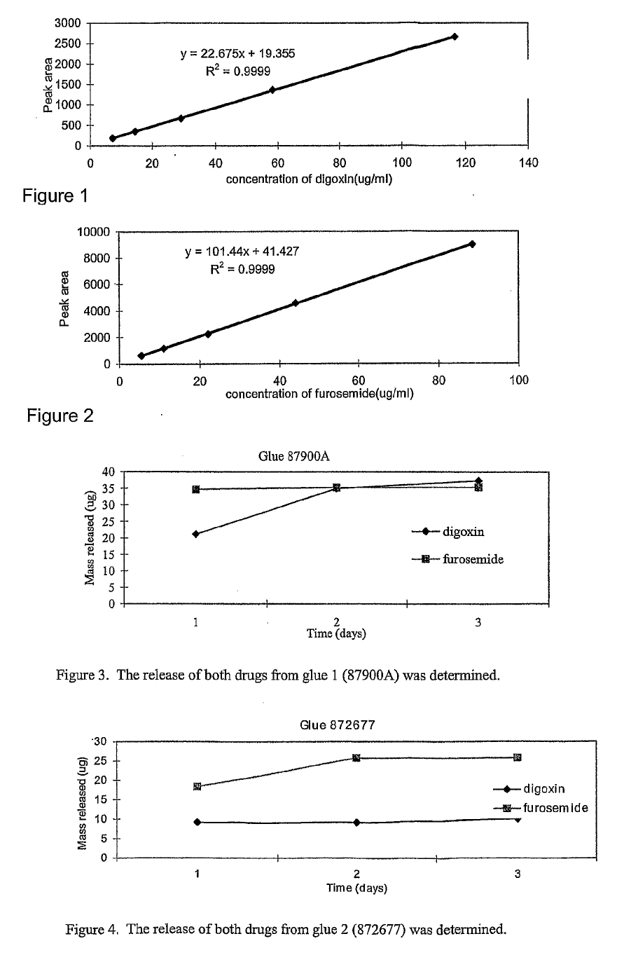

FIG. 1 shows a calibration curve of digoxin concentration according to the HPLC method used.

The HPLC was not able to detect digoxin released from Glue 3 indicating that the digoxin is preferentially bound within this glue.

Glue 1 showed the most favourable release with both drugs releasing at a rapid rate. It was considered that the profile of release indicated that all drug was released over the three day period thus an increased loading of drug within this glue would lead to increased drug release.

FIG. 2 shows a calibration curve of furosemide concentration according to the HPLC method used.

### EXAMPLE 5

### Manufacture of the Delivery Device

Acrylic based pressure sensitive adhesives were sourced from National Starch and Chemical Company with properties that would be appropriate for use with digoxin and furosemide. A study was performed that measure the solubility of the drugs in a range of solvents.

| | | Solubility of Furosemide |
|---|---|---|
| Solvent | Solubility of Digoxin (mg/ml) | (mg/ml) |
| Ethanol | 5.08 | 10.15 |
| Methanol | 8.2 | 15.3 |
| Ethyl acetate | 20.4 | 35.6 |

After mixing the dissolved drug in solvent with glue; a film of 400 μm thickness was cast onto the backing membrane (Scotchpak 1109). This was left uncovered (yet protected from light) for the solvent to evaporate at room temperature for a period of approximately 45 minutes. Once sufficiently dry (approximately 45 minutes) the exposed surface was covered with liner (Stotchpak 1020) to prevent further solvent loss. All materials were cut to a measured size and stored in an airtight container at room temperature. Each patch of known weight had a known drug content, in this case a high loading per surface area is required.

Solvents used in conjunction with drug included, ethylacetate, methanol, ethanol, propanol and combining the dry drug powder with the glue directly.

### EXAMPLE 6

### Measurement of Drug Release from Formulated Patches

Drug release studies were performed as a screening exercise prior to penetration studies. A circular patch of 1 cm diameter of the formulation was taken and placed into a sealed container containing an excess of release medium (2 ml). The vial was sealed and shaken at a controlled speed and temperature (37° C.) for a period of 48 hours. At set time points; 1, 2, 4, 6, 8, 12, 24 and 48 hours a sample (0.5 ml) was removed for analysis. Each time a sample was removed it was replaced with fresh release medium to maintain an overall volume of 2 ml. HPLC analysis of each sample allowed drug release over time to be plotted. The formulations were compared to note those that demonstrate the best release. In the clinical setting the patch will be approximately 0.25 cm² and the release required is 25 μg per 24 hours thus the release rate must be greater than 100 μg/cm²/24 hours.

FIG. 3 shows the release of both drugs from Glue 1 (87900A);

FIG. 4 shows the release of both drugs from Glue 2 (872677);

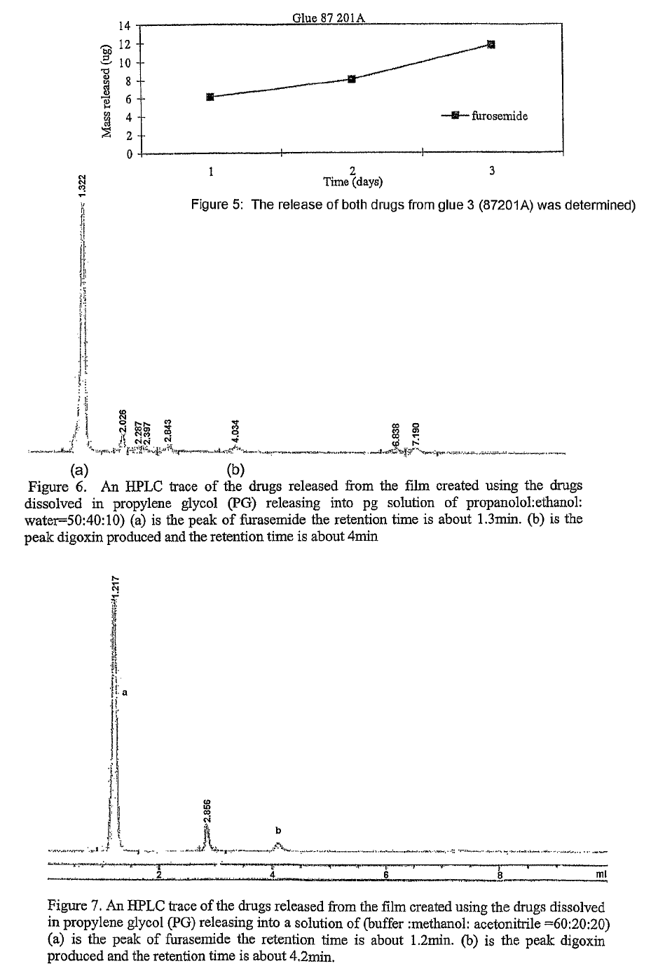

FIG. 5 shows the release of both drugs from Glue 3 (87201A);

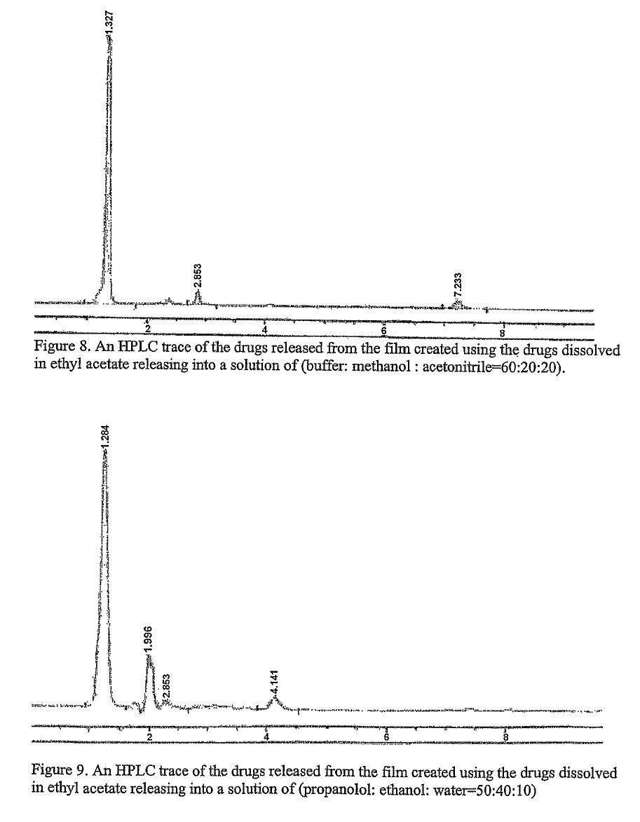

FIGS. 6 to 10 show an HPLC trace of the drugs release from the film in the solvent described releasing into a buffer solution as described.

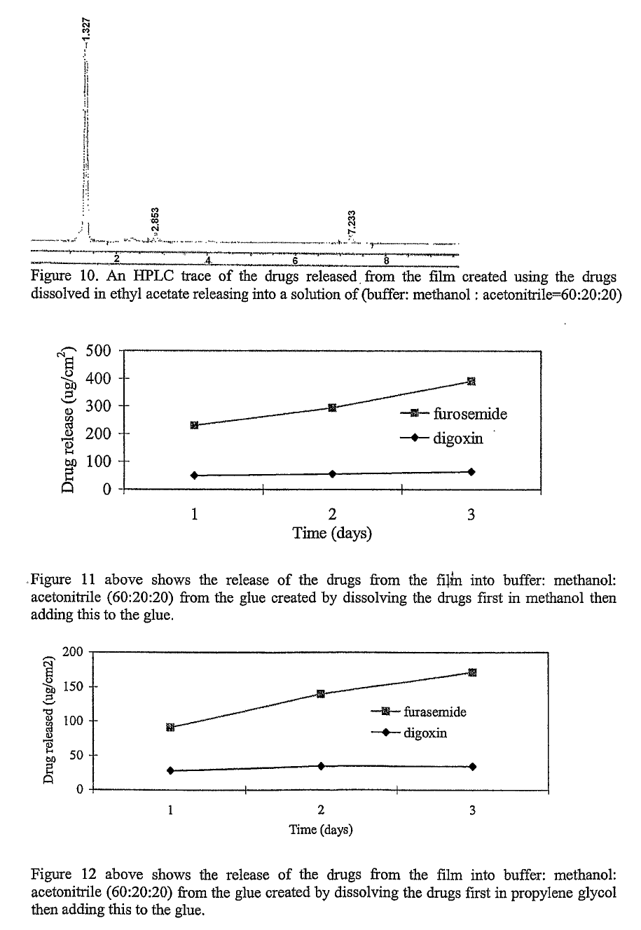

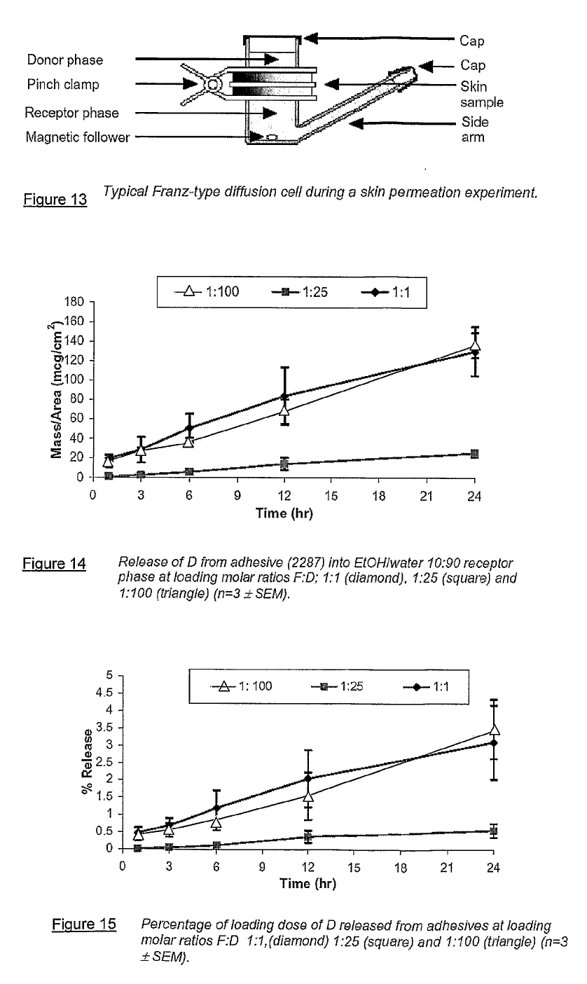

A comparison of the graphs (FIGS. 11 and 12) above show that the drugs are released better when they are formed using methanol to dissolve the drugs rather than propylene glycol.

### EXAMPLE 7

### Measurement of Drug Permeation from Formulated Patches

The pressure sensitive adhesive incorporating the drug that demonstrates the greatest release was selected and the penetration into skin was evaluated. Franz cell apparatus was used to measure the penetration of the drug from the adhesive formulation into the skin membrane.

In the Franz cell, the upper layer represents the transdermal formulation and the lower layer the skin. The vessel below the skin is filled with fluid (the same as used in the release study) and stirred at a constant rate. At designated time intervals a sample from the lower vessel is taken using the side port and analysed using HPLC for drug content. The permeation of drug across the membrane over time can thus be calculated.

The membrane used in this study was a synthetic silicone based skin membrane purchased from Advanced Biotechnologies, USA.

Data from the penetration example suggests that the drug does penetrate the synthetic membrane.

### EXAMPLE 8

### Digoxin and Furosemide Composition

The drug powders were mixed at a 1:1 weight ratio and 500 mg of this mix was blended with 10 mL of Glue 1. This mixture was then cast onto 3M Scotchpak 1020 release liner over an area of 80 by 120 mm. The solvents were left to evaporate and the film was covered with 3M Scotchpak 1109 polyester film laminate backing.

The drug loading is there 2.6 mg/cm² of both drugs within the formulation.

The surface area of the 1 cm diameter patches is 0.785 cm².

Each small patch contains 1.02 mg of digoxin and 1.02 mg of furosemide.

The surface area of the 2 cm diameter patches is 3.142 cm².

Each patch contains 4.08 mg of digoxin and 4.08 mg of furosemide.

### EXAMPLES 9 ET SEQ

The high desirability of >1 dosage form for digoxin and furosemide to address the widely varying anatomical locations of the HPV infection was investigated, proposed variances included:
Plantar warts: drug-in-glue plaster-type application
Hand/finger warts: lacquer/paint

The aim of these later examples is to show both the feasibility of drug-in-glue formulations based on transdermal adhesive and the feasibility of lacquer/paint formulations based upon flexible Collodion BP.

### EXAMPLE 9

### Materials

Digoxin (D) batch number 181104 and furosemide (F) batch number 114310 were obtained from BUFA Pharmaceutical Products by (Vitgeest, Netherlands). Cetrimide lot no. A012633401 was obtained from Acros Organics (New Jersey, USA). Duro-tak® 387-2287 (Glue 4) adhesive was obtained from National Starch and Chemical (Zutphen, Netherlands). Flexible Collodion BP was obtained from J M Loveridge plc (Southampton, UK). HPLC grade acetonitrile, ethanol and methanol were obtained from Fisher Scientific (Loughborough, UK). Pig ears were obtained from a local abattoir, prior to steam cleaning. Water was drawn from an ELGA laboratory still.

### EXAMPLE 9

### Drug-in-Adhesive Formulations

The ratios of F:D selected mix were 1:1, 1:25 and 1:100 (w/w), thus providing a sizeable excess of digoxin. This was based on evidence which suggests that digoxin has substantially greater virostatic power than F (see page 10), indicating that a formulation that delivered an excess of digoxin may be more effective in reducing viral load. The effect each ratio had on the release of digoxin and furosemide is illustrated and ratios investigated which may produce optimum release of each active.

A drug-in-adhesive formulation is a type of matrix system in which drug and excipients can be dissolved or dispersed depending on the amount of drug required for the desired delivery profile (Venkatramann and Gale, 1998). As the solvent in the adhesive evaporates to form a solid matrix product, the concept of thermodynamic activity does not apply. However, we believe, although we do not wish to be bound by any particular theory, the solvent is an important component as it creates microchannels in the matrix upon drying, to form a ‘pathway’ for the drugs to the skin. Generally, the limiting factor in the amount of drug that can be incorporated is the point at which bioadhesive properties are lost.

Preliminary work was performed to refine the composition of the model patches and the method of preparation. A loading dose of 0.5 g of drug mix to 5 g of adhesive was found to be optimum because further addition of drug mix decreased the adhesive properties of the patches. The drug mix was directly added to the adhesive, although 2.5 ml of methanol was added to the mixture in order to decrease viscosity and aid casting out of the patches.

It was determined that to achieve a constant patch thickness, it was preferable to pour the drug-adhesive mixture onto a polymer-lined paper in a horizontal line and then hold the paper vertically allowing the mixture to flow down the paper. This method was found to be reproducible and the drug-in-adhesive covered a surface area of approximately 8 cm² with a depth measured to be almost exactly 1 mm.

### EXAMPLE 10

### Preparation of Drug-in-Adhesive Patches

Patches were prepared by the direct addition of 0.5 g of drug mix, to 5 g of adhesive (wet weight). Three drug mixes were prepared containing different molar ratios of F:D, the compositions of the drug mixes are displayed in Table 1. The appropriate amounts of drug mix and adhesive were accurately weighed directly into glass vials using an analytical balance and 2.5 ml of methanol was added to the mixture. Each vial was vortex-mixed for three minutes and left to rotate on a blood serum rotator overnight, ensuring that the drug mixture was homogeneously dispersed. Control patches were also prepared by the same method, containing no drug mix. Each adhesive mixture was then cast out onto polymer-lined paper as described above. The patches were covered and left for 48 hours to allow the solvent to evaporate (Chedgzy et al 2001). Clear polyethylene film was then attached to the exposed side of the patch to act as patch backing. Individual spherical patches were excised using a cork borer with a diameter of 1 cm (approximately 0.785 cm²).

TABLE 1
Composition of F and D in 0.5 g drug mix - used to prepare patches

| Ratio of F:D | Mass of F (g) | Mass of D (g) |
|---|---|---|
| 1:1 | 0.14885 | 0.35115 |
| 1:25 | 0.0084 | 0.4916 |
| 1:100 | 0.0021 | 0.4979 |

### EXAMPLE 11

### Receptor Phase

The function of a receptor phase is to provide an efficient sink for the released or permeated drug. A rule to which we work is that the amount of drug should not exceed 10% of its solubility in a given sink. Furthermore, the sink must not interfere with the release or permeation process (Heard et al, 2002). Two receptor phases were considered in this work. These were aqueous cetrimide 30 mg/ml, an ionic surfactant and EtOH/water 10:90 v/v, chosen as both drugs are known to be freely soluble in each medium.

Stock solutions of each were prepared in a volumetric flask and degassed by drawing through a 0.45 membrane before use. However, it was subsequently found that cetrimide interfered significantly with the HPLC analysis and for the rest of this work EtOH/water 20:90 v/v was used as a receptor phase.

### Diffusional Release of D and F Mix from Example 10 Patches

The polymer-lined paper was prized from the patches to expose one side of the patch. Each patch was then individually immobilised to the bottom of a general 7 ml glass screw cap vial with a small daub of Glue 4 to the polymer film and allowed to dry for 30 minutes. The dissolution media used were cetrimide 30 mg ml⁻¹ or EtOH/water 10:90 v/v, 5 ml, of each was added individually to each vial. The vials were then placed on a Stuart Scientific Gyro-Rocker (Fisher, UK) set at 70 rpm to ensure adequate mixing of the dissolution medium and incubated at 32° C. (the temperature of the skin) in a laboratory incubator (Genlab). At time points of 1, 3, 6, 12 and 24 hr, (expected period of application) 0.5 ml of dissolution medium was sampled and placed in HPLC auto sampler vials. After each sample was taken, the receptor phase was replenished with 0.5 ml of stock dissolution medium also at 32° C. The samples were refrigerated at 2-4° C. until HPLC analysis 24 hrs later. A total of 3 replicates were performed for each treatment in each receptor phase. The formulation that demonstrated the optimum release was used during permeation examples.

### Rationale for Membrane Selection

To investigate novel topical formulations for treating warts, the delivery of across human wart tissue would be the most appropriate in vitro model. However, such material was not available and so an appropriate model was required. The use of pig skin as a suitable substitute has been demonstrated in several works, with the ear being the part that provides the closest permeability characteristics to human skin (Dick and Scott, 1992; Simon and Maibach, 2000). Permeation experiments were used to study this dermatological drug delivery system, because permeation can predict localisation (percutaneous absorption in the basal layer) the greater the flux, the greater the permeation through the stratum corneum including keratinocytes, which are of greater number in warts than healthy skin. Wart lesions are relatively more keratinised compared to ‘normal’ skin. However, determination of permeation across normal skin could be predictive of permeation through warts, particularly in a screening mode. This is justified as there is some evidence that keratin in skin plays an important part in determining rates of skin permeation (Hashiguchi et al, 1998; Heard et al, 2003).

Freshly slaughtered pigs are routinely subjected to sterilisation by steam cleaning, which has the effect of removing the entire epidermis. The pig ears used in this work were obtained prior to steam cleaning, with epidermis and stratum corneum intact.

### EXAMPLE 12

### Preparation of Pig Ear Skin

The ears were washed under running water and full-thickness dorsal skin was separated from the cartilage via blunt dissection using a scalpel, then hair was removed using an electric razor. The skin was cut into samples of approximately 2 cm² and visually inspected to ensure that each piece was free from abrasions and blood vessels. Specimens were then stored in a crease free state on aluminium foil at −20° C. until required.

### EXAMPLE 13

### Permeation of D and F Mix Across Pig Ear Skin from Patches

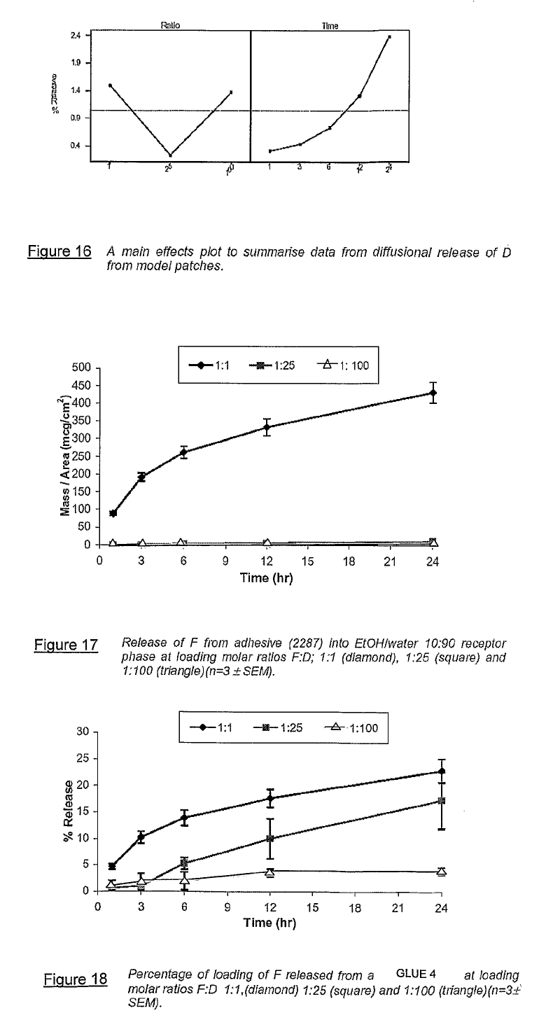

The skin samples were removed from the freezer and left to fully defrost. The donor and receptor compartments of Franz-type diffusion cells (see FIG. 13) were greased, to provide a tight seal and prevent any leakage from the receptor phase. The polymer-lined paper was removed from the patches to expose one side and firmly pressed centrally onto the surface of each piece of skin. After adhesion was established, the skin was mounted onto the flange of a receptor compartment (nominal volume 2.5 ml) of the diffusion sell, ensuring that the patch was placed directly over the flange aperture. The donor compartment was then placed on top and clamped to the receptor compartment using a pinch clamp. EtOH/water 10:90 receptor phase (maintained at 37° C.) was used to fill the receptor compartment carefully to ensure that no air bubbles were in contact with the underside of the skin and the receptor phase was in contact with the skin. A small magnetic stirrer was added to ensure homogeneous mixing of the receptor phase. The Franz cells were placed on a magnetic stirrer immersed in a water bath (containing vercon) and maintained at a constant temperature of 37° C. (therefore the surface of the skin was approximately 32° C.). The donor aperture was occluded to mimic the backing layer of a commercial patch protecting it from moisture and the sampling arms were occluded to prevent evaporation of the receptor phase. At time points of 3, 6, 12, 24, 48 hours, 0.2 μll of receptor phase was sampled and transferred into auto sampler vials which were refrigerated at 2-4° C. until required for analysis. The receptor phase was then replenished. The total number of replicates for each treatment was 5.

### Selection of Paint Medium

Of the array of vehicles available for the topical administration of D and F, a paint-like or lacquer formulation was considered particularly attractive for the treatment of common and genital warts. This is because such treatments are relatively simple and offer a degree of resistance to abrasion. Also, such products are currently commercially available, for example, Salicylic Acid Collodion BP.

### EXAMPLE 14

### Collodion Formulation

Commercially prepared Collodion BP is a liquid, with a high solvent content (mainly diethyl ether). On application to the skin the volatile components of the Collodion rapidly evaporate transforming the liquid solution into a dry, solid film which will adhere to the skin. As with drug-in-glue adhesives, the change in physical state of the vehicle means that the thermodynamic activity, of liquid/semi-solid dermatological systems, only applies to the initial liquid formulation and is irrelevant to the formulation in a solid state. Therefore, the solubility of the actives to a certain extent is arbitrary, as more drug mix can be added by increasing the proportion of solvent to the liquid formulation. After evaporation of the solvents in the formulation on solidification, crystallisation of the compounds will occur however; they will be retained in the matrix of the formulation. This could increase rates of delivery, as direct contact between crystallisation and the skin often provides good delivery, although the precise mechanism of this is unknown. Also affect the ability of the Collodion to maintain intimate contact with the skin at a microscopic level effecting drug delivery i.e. the limiting factor, would be adhesion to the skin.

Several preliminary experiments were conducted to determine the maximum loading of drug mix in Collodion. Problems encountered included sedimentation of drug mix due to limited solubility in Collodion. The drug mix did not easily re-suspend on shaking; meaning that only a small amount of drug mix would dissolve in the Collodion. To overcome this problem, and increase the solubility of the drug mix in Collodion, various amounts of ethanol were added to the formulations until a balance between drug dissolving/reduced rate of sedimentation (which would increase if viscosity decreased) and the rate of drying (solvent evaporating) was found. It was concluded that 0.01 g of drug mix in 5 ml of Collodion and 5 ml of ethanol was a good compromise. This formulation also showed good adhesive properties.

### EXAMPLE 15

### Preparation of Collodion Formulations

Drug mix (for composition see table 2) 0.02 g (a stock was made) was weighted on an analytical balance (accurate to 5 decimal places) and added directly to 10 ml of Collodion and 10 ml of ethanol in a McCartney bottle. The molar ratios used were F:D; 1:1, 1:2.5 (2:5) and 1:10 because a smaller amount of drug mix was used, compared to the drug-in-adhesive and this allowed measurable amounts of F to be used. Each of the McCartney bottles was vortexed for three minutes and left to rotate on a blood serum rotator overnight, to ensure that the mixture was homogeneous and that any air bubbles present had dispersed. Control Collodions were also prepared by the same method, however, no drug mix was added.

TABLE 2
Composition of F and D in 0.01 g drug mix - used to
prepare Collodions.

| Ratio of F:D | Mass of F (g) | Mass of D (g) |
|---|---|---|
| 1:1 | 2.977 × 10⁻³ | 7.023 × 10⁻³ |
| 1:2.5 | 1.447 × 10⁻³ | 8.553 × 10⁻³ |
| 1:10 | 4.058 × 10⁻⁴ | 9.594 × 10⁻³ |

### EXAMPLE 16

### Diffusional Release of D and F from Collodions

Different molar ratios of the two drugs were used to determine affect upon release rate and the extent of the release of each drug. The Collodion, 200 μl, was dispensed to the bottom of general 7 ml glass screw cap vials using a Gilson Pipette and left to dry for three hours. Then 2 ml of dissolution medium, again de-gassed EtOH/water 10:90, was added to each vial. The amount of receptor phase sampled and replenished was 200 μl, with a total of five replicates performed for each treatment. The formulation that demonstrated optimum release was selected for skin permeation experiments.

### EXAMPLE 17

### Permeation of D and F Across Pig Ear Skin from Collodion

The method was essentially the same as described in example 16. Mounted skin membranes were does with 200 μl of Collodion and left for thirty minutes to dry before the receptor phase was added. A total number of four replicates were performed for each treatment.

### High Pressure Liquid Chromatography (HPLC) Analysis

HPLC analysis was performed using the same method as described previously i.e. an Agilent series 1100 automated system, fitted with a Phenomenex Kingsorb 5 mm C18 Column 250×4.6 mm (Phenomenex, Macclesfield, UK) and a Phenomenex Securiguard guard column. D and F were detected using an ultraviolet (UV) detector set at wavelength 220 nm. The mobile phase consisted of 40:30:30 Water:MeOH:MeCN, de-gassed by drawing through a 0.45 membrane and run isocratically for 10 min at a flow rate of 1 ml min⁻¹. The injection volume of each sample was 20 μl. The retention time of F and D was typically 2.6 minutes and 5.2 minutes respectively, (see FIG. 15). Data were acquired using Agilent software. Standard calibration curves were determined using standard solutions of 5, 10, 20, 40, 80 and 100 μg ml⁻¹ in the receptor phase, to prevent solvatochronic effects. The limit of detection was 0.1 μg ml⁻¹.

### Data Handling

Chromatogram peaks were integrated manually, and the data corrected for dilution effects. Cumulative release was determined and plotted against the square route of time to determine release rates. Cumulative permeation data were determined and plotted against time to order to obtain flux. Excel was used for data processing and Minitab for statistical analysis.

### EXAMPLE 18

### Diffusional Release of Digoxin from Patches

### Cumulative Mass of Digoxin Released

Cumulative release (mass/area) profiles of digoxin from adhesive containing molar ratios of F:D; 1:1, 1:25, 1:100 were determined over 24 hr and are illustrated in FIG. 14. Digoxin was released from all the patches. The trend in the greatest cumulative release after 24 hr (table 3) was 1:100>1:1>1:25. The patches containing ratios of 1:1 and 1:100 had similar profiles, and up to 12 hr the greatest release was observed form the patches containing a molar ratio of 1:1. Error bars were small.

Percentage Release of Loading Dose of Digoxin from Model Patches

The percentage release of the loading dose of digoxin from adhesives containing molar of F:D; 1:1, 1:25 and 1:100 was determined over 24 hr and are displayed in FIG. 15. The percentage release mimics the trend observed in FIG. 14. Maximum percentage release values of digoxin after 24 hr are illustrated in table 3. Error bars were small.

TABLE 3
Maximum release values of digoxin from patches at 24 hr

| Ratio | Q₂₄ release Mass/Area (μg/cm²) | Q₂₄ release % |
|---|---|---|
| 1:1 | 130.03 | 3.17 |
| 1:25 | 25.25 | 3.49 |
| 1:100 | 136.18 | 0.56 |

### EXAMPLE 19

### Main Effects Plot Illustrating Digoxin Release Data from Patches

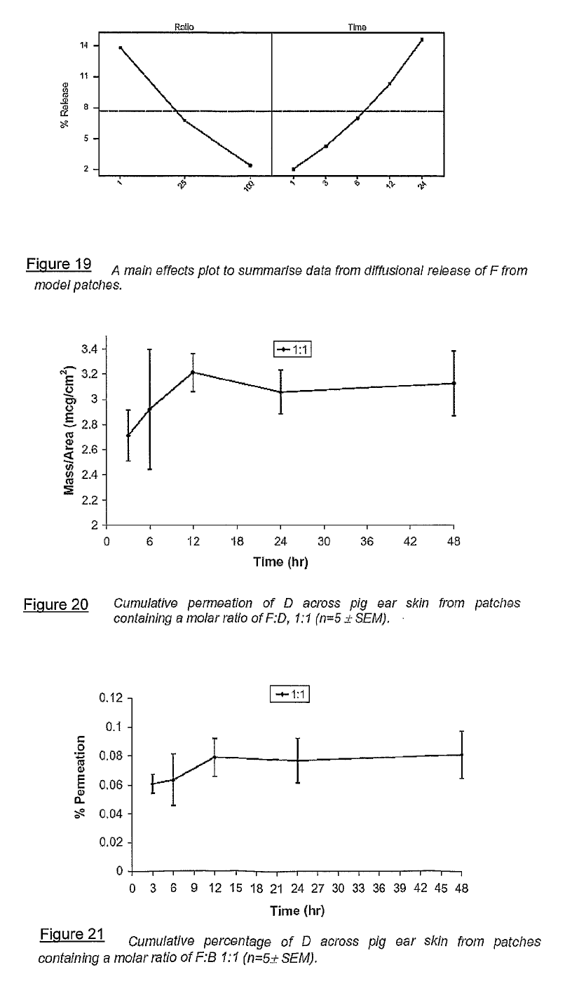

The main effects plot illustrated in FIG. 16 used to visually summarise the data from the diffusional release of digoxin from model patches. It illustrates the trend in ratio of percentage release of the loading dose of digoxin and how this increases over time.

### EXAMPLE 20

### Determination of Rate of Release (of Loading of) Digoxin from Patches

Linearity denoted by the cumulative release (mass/area) profiles in FIG. 14 indicated zero order release kinetics from all three molar ratios. Rate of release was determined from the gradient of a trend line for each profile. For ideal linearity R²=1. Release values are illustrated in table 4.

TABLE 4
Release rate of digoxin from model patches and R² values
for each molar ratio

| Ratio | Release rate (mcgcm⁻² h⁻¹) | R² |
|---|---|---|
| 1:1 | 4.8353 | 0.9858 |
| 1:25 | 1.0844 | 0.9916 |
| 1:100 | 5.1899 | 0.9945 |

### EXAMPLE 21

### Diffusional Release of Furosemide from Model Patches

### Cumulative Mass of F Released

Cumulative release (mass/area) profiles of F from adhesive containing molar ratios of F:D of 1:1, 1:25, 1:100 were determined over 24 hr and are illustrated in FIG. 17. Furosemide is released from all the patches. The 1:1 ratio demonstrates a typical release profile, whereas release from 1:25 and 1:100 is linear. The trend in greatest cumulative release after 24 hrs was 1:1>1:25>1:100 (see table 3.3 for maximum release values). Error bars were small.

### EXAMPLE 22

### Percentage Release of Loading Dose of Furosemide from Model Patches

The trend in percentage release of loading dose of furosemide (FIG. 18) mimics the trend observed in 3.6, for maximum percentage release after 24 hr refer to table 5. Error bars were small.

TABLE 5
Maximum release values of Furosemide from model patches at 24 hr

| Ratio | Q₂₄ release Mass/Area (μg/cm²) | Q₂₄ release % |
|---|---|---|
| 1:1 | 432.02 | 22.82 |
| 1:25 | 10.77 | 17.23 |
| 1:100 | 2.85 | 3.85 |

### EXAMPLE 23

### Main Effects Plot to Illustrate Release Data of Furosemide from Patches

The main effects plot illustrated in FIG. 19 summaries the data from the diffusional release of furosemide from model patches. It illustrates the trend in ratio of percentage release of loading dose of F and how percentage release of loading of furosemide increased over time.

### EXAMPLE 24

### Permeation of Digoxin and Furosemide Mix Across Pig Ear Skin from Patches

Permeation of Digoxin Across Pig Ear Skin from Patches

Permeation of digoxin across pig skin is illustrated as both cumulative mass/area and percentage permeation of loading of digoxin and is shown in FIGS. 20 and 21 respectively. The profiles are of a similar shape and are atypical permeation profiles. However, they do illustrate that digoxin has permeated the pig skin. Error bars are larger than for release results. Apparent maximum flux (table 6 along with maximum permeation values) was calculated from FIG. 21 however lag time and Kp could not be calculated from these profiles.

### EXAMPLE 25

### Permeation of Furosemide Across Pig Ear Skin from Patches

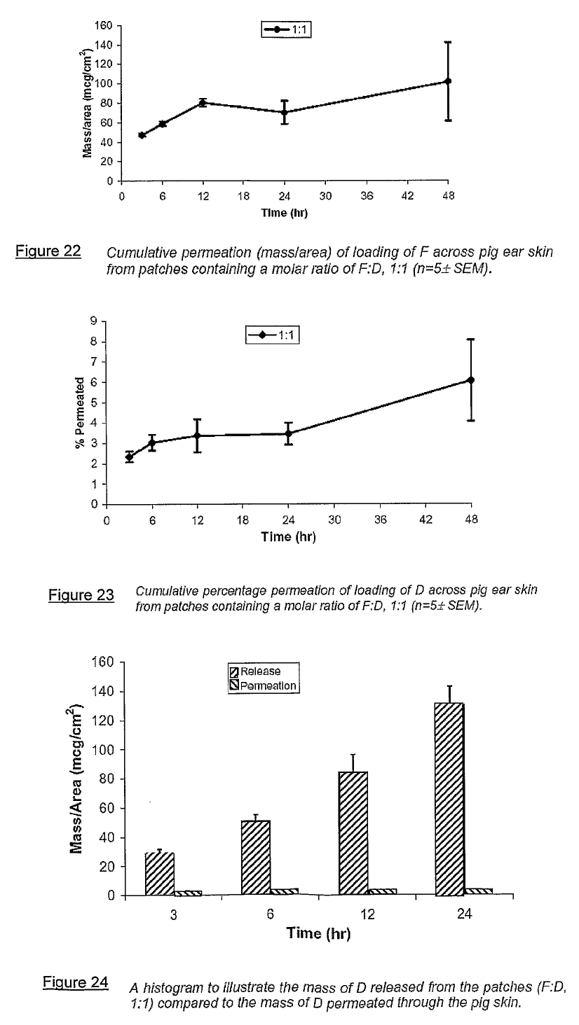

Permeation of furosemide across pig skin is illustrated as both cumulative release (mass/area) of loading and percentage permeation of loading of furosemide and is shown in FIGS. 22 and 23 respectively. Both of the profiles are of a similar shape and are atypical permeation profiles. However, they do show that furosemide has permeated the pig skin. Error bars are larger than for release and permeation of digoxin across pig skin. Apparent flux maximum (table 6 and maximum permeation values) was calculated, however lag time and Kp could not be calculated from FIG. 22.

TABLE 6
Maximum permeation values of digoxin and furosemide from patches
across pig skin

| | Q₂₄ permeation | | | |
|---|---|---|---|---|
| | Mass/Area | Q₂₄ | Apparent flux | |
| Active | (μg/cm²) | permeation % | maximum μgcm⁻² h⁻¹ | SEM |
| F | 101.92 | 6.07 | 0.158 | 0.072 |
| D | 5.81 | 0.12 | 3.499 | 0.372 |

### EXAMPLE 26

### Comparison Between Mass Released from the Patches Containing a F:D in a 1:1 Ratio and Mass Permeated Through the Skin

Comparison Between the Mass/Area of Digoxin Released from the Patches and Mass/Area of Digoxin that Permeated the Skin

FIG. 24 illustrates the mass/area of digoxin released from the patches and also the mass/area of digoxin that permeated the skin and allows a comparison to be made. A larger mass of digoxin was released from the patches that permeated the skin.

### EXAMPLE 27

### Comparison Between the Mass/Area of Furosemide Released from the Patches and Mass/Area of Furosemide that Permeated the Skin

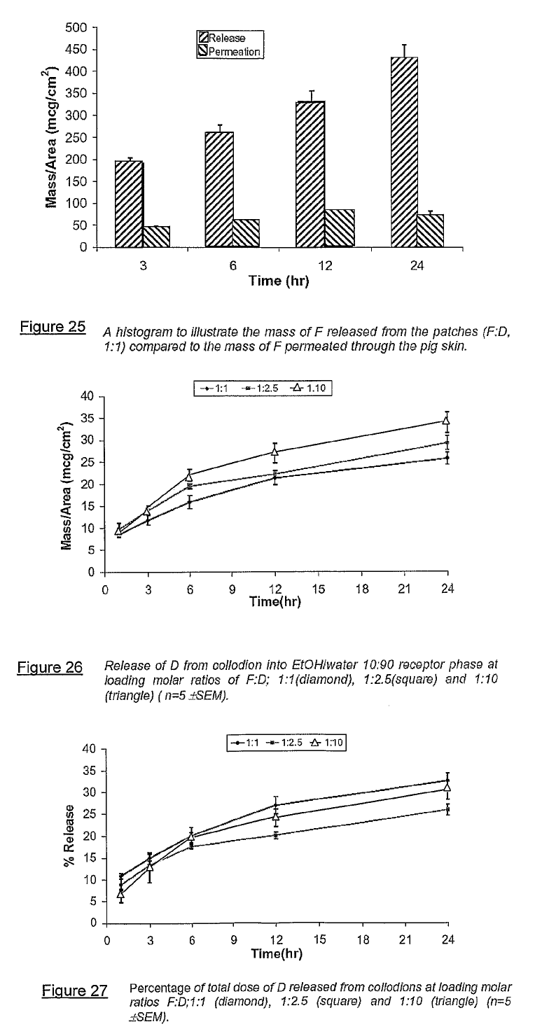

FIG. 25 illustrates the mass/area of furosemide released from the patches and also the mass/area of furosemide that permeated the skin and allows a comparison to be made. A larger mass of furosemide was released from the patches that permeated the skin.

### EXAMPLE 28

### Diffusional Release of Digoxin from Collodion

Cumulative Mass/Area of Digoxin Released from Collodions

Cumulative release profiles of digoxin from Collodions containing molar ratios of F:D, 1:1, 1:2.5 and 1:10 were determined over 24 hr and are illustrated in FIG. 26 released from each of the Collodions. The trend the in greatest cumulative release after 24 hr (see table 7) was 1:100.1:2.5>1:10. The shape of the three profiles were similar and error bars small.

### EXAMPLE 29

### Percentage Release of Loading Dose of Digoxin from Collodion

The percentage release of the loading dose of digoxin from Collodions containing molar ratios of F:D; 1:1, 1:2.5 and 1:10 was determined over 24 hr and are displayed in FIG. 27. The percentage release mimics the trend observed in FIG. 26. Maximum percentage release values of digoxin after 24 hr are illustrated in table 8. Error bars were small.

TABLE 8
Maximum release values of digoxin from Collodions after 24 hr

| Ratio | Q₂₄ release Mass/Area (μg/cm²) | Q₂₄ release % |
|---|---|---|
| 1:1 | 25.78 | 32.54 |
| 1:2.5 | 29.32 | 25.89 |
| 1:10 | 34.01 | 30.36 |

### EXAMPLE 30

### Determination of Rate of Release of Loading of Digoxin from Collodion

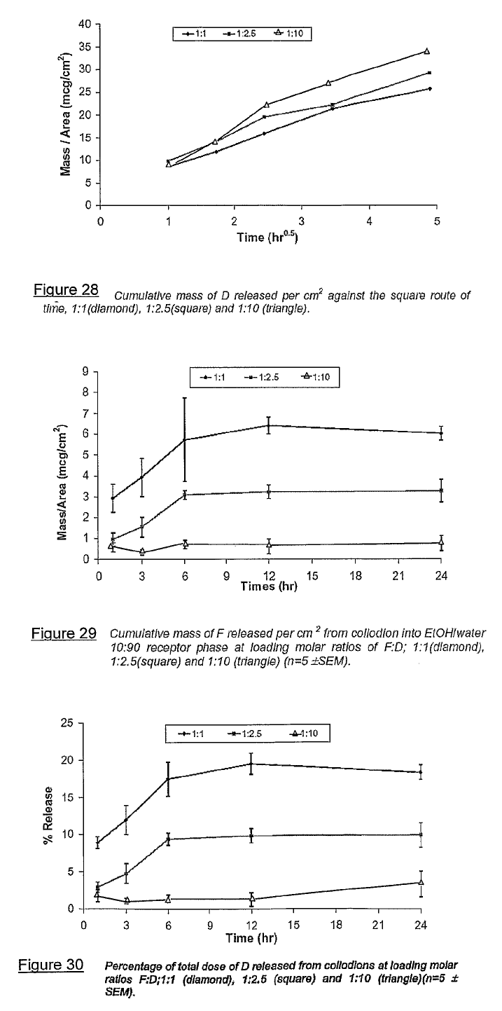

FIG. 28 illustrates the cumulative release of digoxin from the three different Collodions plotted against the square root of time. Linearity of the plots indicates first order release kinetics, 1:10 shows the greatest rate of release. R² and rate of rate of release are illustrated in table 9.

TABLE 9
Rate of release values of digoxin from Collodion

| Ratio | Release rate (mcgcm⁻² h^{-0.5}) | R² |
|---|---|---|
| 1:1 | 4.5393 | 0.9859 |
| 1:25 | 4.8852 | 0.9816 |
| 1:100 | 6.5231 | 0.9709 |

### EXAMPLE 31

### Diffusional Release of Furosemide from Collodion

Cumulative Mass/Area Released of Furosemide from Collodion

The cumulative release profiles of furosemide from Collodions containing molar ratios of F:D; 1:1, 1:2.5 and 1:10 were determined over 24 hr and are shown in FIG. 29. Furosemide is released from all the different Collodions producing a typical release profile. The trend in greatest cumulative release after 24 hr was 1:1>1:2.5>1:10 (see table 10 for maximum release values). The size of the error bars varied.

### EXAMPLE 32

### Percentage Release of Loading Dose of Furosemide from Collodions

The trend in percentage release of loading dose of furosemide (FIG. 30) mimics that of cumulative release. For maximum percentage release after 24 hr see table 10. Error bars were small.

TABLE 10
Maximum release values of furosemide from Collodion after 24 hr

| Ratio | Q₂₄ release Mass/Area (μg/cm²) | Q₂₄ release % |
|---|---|---|
| 1:1 | 6.02 | 18.33 |
| 1:2.5 | 3.27 | 9.95 |
| 1:10 | 0.77 | 3.33 |

### EXAMPLE 33

### Release Rates of Furosemide from Collodion

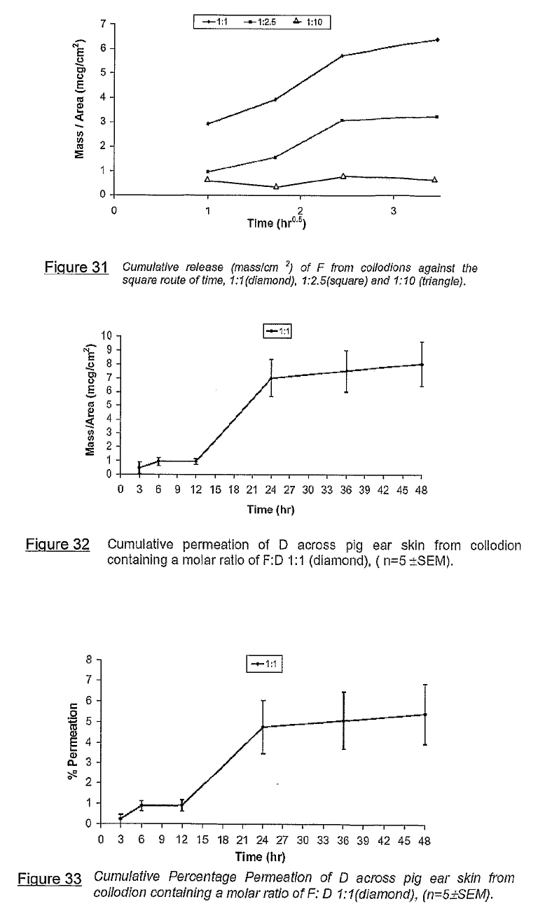

FIG. 31 depicts cumulative release of furosemide from the Collodions containing the three different molar ratios plotted against the square root of time. Linearity was reported from reported from 1:1 indicating first order kinetics. For release values refer to table 11.

TABLE 11
Rate of release data of furosemide from Collodion

| Ratio | Release rate (mcgcm⁻² h^{-0.5}) | R² |
|---|---|---|
| 1:1 | 1.4811 | 0.9438 |
| 1:2.5 | 1.0043 | 0.8742 |
| 1:10 | 0.0575 | 0.1356 |

### EXAMPLE 34

### Permeation of Digoxin and Furosemide Mix Across Pig Ear Skin from Collodions

Permeation of Digoxin Across Pig Ear Skin from Collodions

Permeation of digoxin across pig skin is illustrated as both cumulative mass/area and cumulative percentage of loading of digoxin and are illustrated in FIGS. 32 and 33 respectively. Both of the profiles are similar in shape and are atypical of permeation profiles. However they do illustrate that digoxin from Collodion permeates through the skin. Error bars were larger than for Collodion release results. For AFM and maximum permeation values refer to table 12. Lag time and Kp could not be calculated from these profiles.

### EXAMPLE 35

### Permeation of Furosemide Across Pig Ear Skin from Collodion

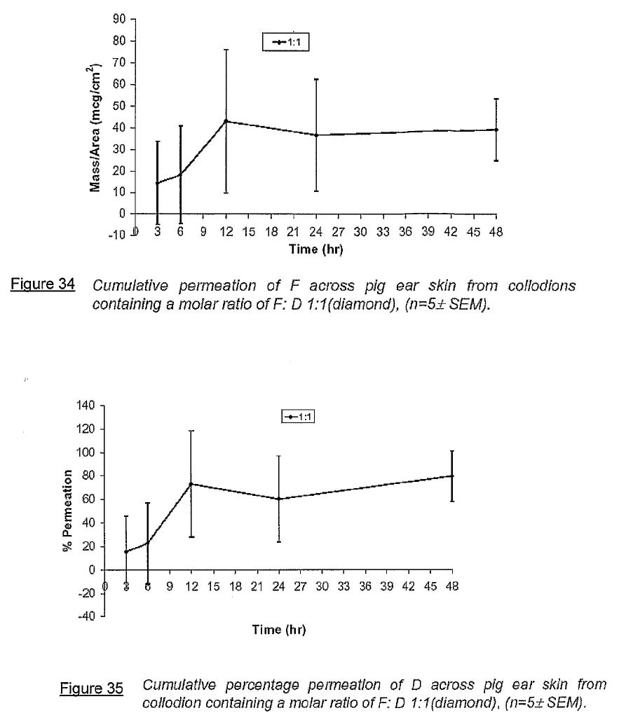

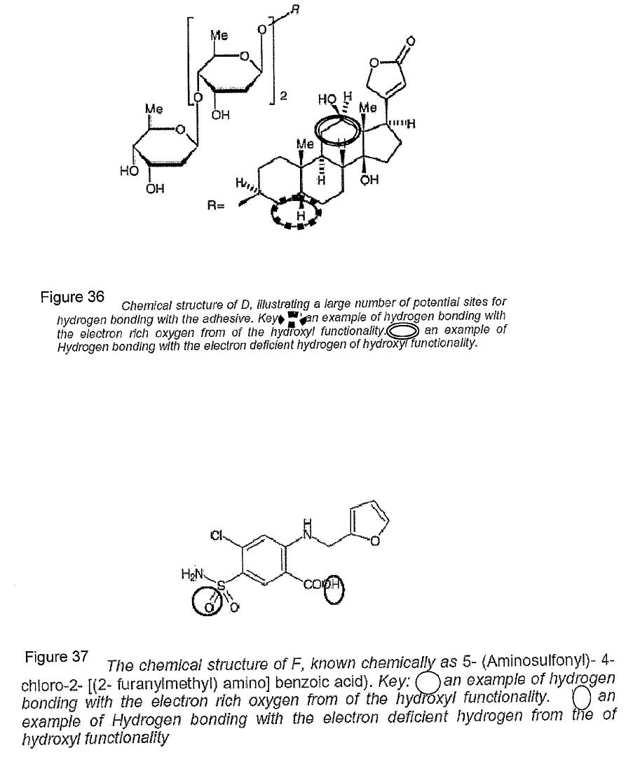

Permeation of furosemide across pig ear skin is illustrated as both cumulative mass/area and cumulative percentage and shown in FIGS. 34 and 35 respectively. The profiles are of a similar shape and are atypical permeation profiles. However, they do show that furosemide permeated the pig skin. Error bars are large. AFM and maximum permeation values are displayed in table 12. However, lag time and Kp could not be calculated from FIG. 34.

TABLE 12
Maximum permeation values of digoxin and furosemide mix from
Collodion

| | Q₂₄ permeation | | | |
|---|---|---|---|---|
| | Mass/Area | Q₂₄ | Apparent maximum | |
| Active | (μg/cm²) | permeation % | flux μgcm⁻² h⁻¹ | SEM |
| F | 39.45 | 79.64 | 4.3423 | 2.05 |
| D | 8.03 | 5.39 | 0.313 | 0.83 |

### EXAMPLE 36

### Comparison Between Mass Released from the Collodion Containing F:D in a 1:1 Molar Ratio and Mass Permeated Through the Pig Skin

### Controls

Controls were used throughout this work. During the release studies, formulations containing no actives were used as controls. The corresponding chromatograms illustrated no peaks at the wavelength of detection.

During permeation studies formulations containing no actives and skin without a formulation applied to it were used as controls. The corresponding chromatograms illustrated no peaks at the wavelength of detection.

Diffusional Release of Digoxin and Furosemide from Patches

Dermatological formulations are required to release the active compound(s) at the surface of the skin. Generally, it is thought that the rate-limiting step in skin permeation is transport across the stratum corneum, although in some cases the rate-limiting step can be release of the active compound(s) from the formulation. If this occurs the bioavailability of the compound(s) may be affected. This is less likely to happen during the permeation of digoxin and furosemide through callous wart material. Warts contain a greater proportion of keratinocytes compared to normal skin, which can modulate the extent, and rate of absorption.

The release of digoxin and furosemide from the adhesive could potentially be limited by three parameters:molar ratio, drug loading and the interaction of the drugs with adhesive. The aim of this investigation was to establish which molar ratio would release the maximum mass of digoxin and a sufficient mass furosemide and could therefore be used in subsequent permeation studies. Overall the release of digoxin would have a greater influence in the choice of ratio than furosemide, refer to Example 14.

Diffusional Release of Digoxin from Patches

These results showed that a proportion of the loading mass of digoxin was released from all of the patches. The extent of release was observed in terms of cumulative release (mass/area), to establish the maximum mass/area of digoxin released. From this the maximal dose that could potentially come in contact with the surface of the patients' skin could be estimated. This was found to be in the order of 136.18 μgcm⁻². An initial burst in the release of digoxin was observed from all of the patches. This was most prominent from the patches containing 1:1 and 1:100 molar ratio. This may be due to release of digoxin molecules at or near the surface of the patch. The release from all three ratios was linear, displaying zero order release kinetics, which are desirable of a topical delivery device. The trend for greatest release (mass/area) was 1:100>1:1>1:25. The 1:100 ratio gave the greatest mass/area released as expected because it contained the largest mass/area of digoxin. The 1:1 ratio gave similar results, which was not expected as it contained the smallest mass of digoxin, suggesting that loading, was not the rate-limiting factor of release.

Percentage release of the loading dose was calculated to allow, for slight variation in patch preparation, and comparison between the formulations. Percentage release was expected to be small with a large amount of drug retained in the matrix.

The trend observed in percentage release of loading was the same as for cumulative release (mass/area). Differences observed in the percentage release of loading, from each formulation indicated that percentage release was not proportional to drug loading. Otherwise the percentage release from each formulation would be the same.

Statistical evaluation performed by a two-way ANOVA indicated that there was a significant difference in percentage release of loading of digoxin, between 1:25 and the other ratios. A significant difference in percentage release at each time point was also illustrated and increased with time. This suggests that a substantial proportion of digoxin was still being released after 24 hr. In clinical practice, regarding the delivery of digoxin, the patch would not have needed to be changed within this time period. Thereby reducing frequency of administration and consequently increasing patient compliance.

The rate of release was examined, in order to distinguish between 1:1 and 1:100 in terms of which formulation would give the maximum delivery of D in the shortest time period. Although the rate of release from 1:100 was the greatest at 5.19 μg cm⁻² h⁻¹ it was surprisingly similar to that of 1:1 at 4.84 μg cm⁻² h⁻¹.

Diffusional Release of Furosemide from Patches

A proportion of furosemide was released from all the patches and this confirmed that both drugs were released simultaneously from the matrix and therefore could potentially simultaneously permeate the skin.

Again the extent of release was observed as cumulative release (mass/area) to establish the maximum mass released, and hence the maximal dose of furosemide that could potentially come into contact with a patient's skin. This was found to be in the order of 432.02 μg cm⁻².

No initial burst in release of furosemide was observed, suggesting that furosemide was uniformly distributed in the matrix. The trend in release was 1:1>1:25>1:100. The 1:1 ratio gave a typical release profile, demonstrating depletion of furosemide after 3 hr, and greater cumulative release of furosemide than the other ratios. Although this was expected as 1:1 contained the greatest mass of furosemide, the difference in magnitude of release from the other ratios was unexpected. The 1:25 and 1:100 ratios gave linear release profiles illustrating desirable zero release kinetics.

### Percentage Release of Loading Followed the Same Trend as Cumulative Release.

Percentage release ranged from 22.82% (1:1)-3.85% (1:100), illustrating relatively high percentage release of F from 1:1. Overall the percentage release values for furosemide were greater than those obtained for digoxin.

Statistical evaluation by a two-way ANOVA, indicated that there was a significant difference between 1:1 and the other ratios. The main effects plot illustrated that optimum percentage release was obtained from 1:1, which also released a greater mass/area. A significant difference in percentage release at each time point (also seen with digoxin) was shown via the main effects plot, to increase over time, concluding that frequency of administration of these patches would be at the most once every 24 hr.

### Error Bars Indicating Good Reproducibility Between Samples.

Huguchi, (1962) stated that drug release from matrix devices such as patches is often a function of the square root of time. Linear plots indicate first order release kinetics. For the 1:1 ratio it was necessary to plot cumulative release (mass/area) against the square root of time in order to establish order and rate of reaction as cumulative release (mass/area) did not indicate zero order release kinetics. Although the 1:1 ratio exhibited first order release kinetics, the rate of release was much greater and the mass/area released was considerably larger than for the other ratios, suggesting that the 1:1 ratio was the prime choice in terms of furosemide delivery.

In summary, this data provided sufficient information to allow the rational selection of the most promising formulation for permeation studies. Thus patches containing D:F in a 1:1 molar ratio were selected. Percentage release of both digoxin and furosemide is greater than from the other ratios. The 1:1 ratio also released the greatest mass/area of both drugs.

The larger the concentration gradient, the higher the rate of permeation. This ratio also provided the greatest rate of release i.e. an optimal mass is released in the shortest time.

Permeation of Digoxin and Furosemide Mix Across Pig Skin from Model Patches Containing 1:1 Molar Ratio

Dermal absorption involves several processes. Firstly the actives are released from the formulation; they then encounter the surface of the skin and establish a stratum corneum reservoir. This leads to penetration of the barrier and finally diffusion into another compartment of the skin (Schaefer and Redelmeler, 1996).

Permeation profiles were presented as cumulative mass/area and cumulative percentage permeation of total loading. Cumulative permeation results illustrated that both digoxin and furosemide permeated the skin and therefore have potential as a future localised antipapillomavirus treatment. Permeation through the skin can predict localisation and therefore it is possible that both digoxin and furosemide are coming in to contact with the basal layer of the epidermis.

Comparison Between the Mass of Digoxin and Furosemide Released from Model Patches Containing F:D 1:1 and Mass Permeated Through the Skin

Differences were observed in the mass/area of digoxin and furosemide released from the patches and the mass/area of digoxin and furosemide permeated across the skin, in that mass released was greater than that permeated. Assuming that the mass released of digoxin and furosemide from the patches into the dissolution medium is approximately the same as that released at the stratum corneum. This suggests that a quantity of the each of the actives could be retained in the skin. From visual inspection of FIGS. 26 and 27 it is possible to observe that a higher proportion of digoxin than furosemide is retained in the skin. This was a positive result as it is desirable to have an excess of digoxin at the site of infection.

Diffusional Release of Digoxin and Furosemide from Collodion

As with the patches, the release of digoxin and furosemide from the Collodion could be potentially limited by three parameters, molar ratio, drug loading and interaction between the drugs and the Collodion matrix. The aim of this experiment was to establish which Collodion contained the molar ratio of D:F that released the maximum amount of digoxin and a sufficient amount of furosemide. This would be used for further permeation studies. Overall the release of digoxin would have a larger influence in choice of ratio over release of furosemide (Example 14).

Diffusional Release of Digoxin from Collodion

The results illustrated that a proportion of the loading mass of digoxin was released from all three of the Collodions, and release increased over time. Cumulative release (mass/area) plots depicted extent of release and illustrated the maximum dose released after 24 hr. The maximal dose of digoxin released after 24 hr was in the order of 34.01 μg cm⁻² and is, in theory, the dose delivered to the surface of the patients' skins.

Cumulative release (mass/area) profiles for the three ratios, were typical of release, and began to plateaux after six hours. The trend for release was 1:10>1:2.5>1:1, and was expected demonstrating a proportional relationship between the initial mass of digoxin in the Collodion and the mass released from it. From these results it is possible that loading mass, molar ratio or interaction with the vehicle (Collodion) could be the limiting factor in mass released.

Release profiles for percentage release of loading dose were also plotted, to allow for variation in volume of Collodion pipette into each vial and to allow comparison between formulations. Percentage release ranged from 25.54-30.36%, which was relatively high compared to approximate 10%, expected and compared to the patches. This suggested that differences between the adhesive and Collodion matrix could be responsible. A possible explanation could be the formation of larger micro channels in the matrix of the Collodion as the solvent evaporates on drying, or a greater number may be formed than in the patches due to the higher solvent content of Collodion.

Percentage release of loading dose did not follow the same trend as cumulative release mass/area, and instead was 1:1>1:10>1.2.5. However, this trend correlated with the trend in cumulative mass/area released of digoxin from the patches. This suggested that the effect of the vehicle would only have an influence on the over all extent of release from all three of the Collodions, and that the difference in molar ratios contribute towards the trend.

Statistical evaluation by a two-way ANOVA, illustrated that there was a significant difference between 1:1 and the other ratios. Optimum percentage release was attained from 1:1, however this did not give the largest mass/area released. A significant difference in percentage release at each time point was observed (as with digoxin) which increased over time, concluding that frequency of administration of the Collodions for the delivery of digoxin, like the patches would be at the most once every 24 hr.

Error bars were small indicating good reproducibility between samples. In summary at this stage of the investigation, likewise with the patches the decision of which Collodion will be used for permeation studies lay between 1:1 and 1:10 (i.e. the lowest and greatest excess moles of digoxin).

Linear plots indicated first order release kinetics. In general the rates of release were similar, although 1:10 gave the greatest rate of release whilst 1:1 gave the smallest, the optimum molar ratio could not be determined from this data.

Diffusional Release of Furosemide from Collodion

Furosemide was released form all the Collodions, indicating that all the Collodions could be potentially used in permeation studies, as they illustrated simultaneous release of digoxin and furosemide. Maximal dose released after 48 hr was in the order of 6.02 μg cm⁻².

Cumulative release (mass/area) of furosemide from Collodion was lower than that of digoxin, unlike the patches, thereby potentially delivering more of digoxin to the site of infection, which was desirable. The profiles from all the molar ratios were typical of release, an initial burst was observed between 1-6 hr, and plateau in the profile at 6 hrs, which was comparable with the digoxin release profiles. This was most likely to be due to depletion, because it was observed from both drugs and to a lesser extent in the patches (which contained a higher dose of digoxin and furosemide). The trend in cumulative release (mass/area) was 1:1>1:2.5>1:100 and was unexpected as 1:1 contained the lowest (mass/area) of furosemide. This trend was also observed in the percentage release data which indicates that digoxin having an effect on the release of furosemide as otherwise one would expect the percentage release of furosemide to be the same for each ratio.

Statistical evaluation by a two-way ANOVA, indicated a significant difference between 1:1 and the other ratios. Optimum percentage release was obtained from 1:1, which also released the greatest mass. A significant difference in percentage release at each time point was illustrated as with digoxin, less of an increase as observed within time points after 6 hr. This suggests that administration of Collodion may be required more frequently for optimum delivery of furosemide.

Error bars throughout this part of the investigation were small indicating good reproducibility between samples. In summary of this data, for delivery of F, the 1:1 ratio appeared to be the strongest candidate.

Cumulative mass/area released of furosemide against the square root of time, depicted linearity for 1:1 ratio with R² value close to 1. This ratio also illustrated the highest rate of release. However R² values for the other ratios were riot close to 1 indicating poor correlation.

Comparison Between Digoxin and Furosemide Release Data from Collodion

In summary, a decision of which ratio would potentially provide optimum delivery of digoxin and furosemide was not as clear as for the patches, especially regarding the release of digoxin.

This investigation provided enough information for a molar ratio to be chosen for permeation studies. Patches containing D:F in a 1:1 molar ratio were used as, percentage release of both digoxin and furosemide was essentially greater than from the other ratios. The 1:1 ratio also released the greatest mass/area of furosemide. Providing the greatest concentration gradient.

Permeation of Digoxin and Furosemide Across Pig Skin from Collodion Containing Digoxin and F in a 1:1 Molar Ratio

Permeation data was shown as cumulative mass/area and percentage permeation of total loading. The permeation data illustrated that both furosemide and digoxin simultaneously permeated the skin, and can be used as a prediction of localisation.

The permeation profiles for both digoxin and furosemide were atypical as were the permeation profiles for the patches. Therefore suggests this could be related to the nature of the actives individually or in combination. The profile for digoxin is however different to that of furosemide differing from a typical profile only during phase 1. The percentage release profile for digoxin mimicked this shape. The profiles for furosemide were a similar shape to that seen from the patches.

The SEM for the permeation profiles was larger in magnitude than those for the release profiles. This indicated less reproducibility in data compared to the release data. The major difference between the release experiments and the permeation was the introduction of the skin, therefore this may have had an impact on the results. The SEM was also of a larger magnitude for furosemide compared to digoxin. A reason for this could be the amount of solvent present in the liquid state of the Collodion (all solvent had evaporated from the patches during preparation) could affect the integrity of the skin and reduce reproducibility between replicates. The number of replicates was 4 compared to five for the patches, which may also have had an impact.

The atypical nature of these profiles meant that SSF could not be accurately measured and AMF was measured instead. For digoxin this was calculated between 12-24 hr to be 0.313 μg cm⁻² h⁻¹ and for furosemide between 6-12 hr to be 4.3423 μg cm⁻² h⁻¹. It was not possible to measure lag time and only an estimation of kp was calculated.

The mass/area of digoxin that permeated the skin was 8.02 μg cm⁻² (1.03×10⁻⁸ μg cm⁻²) compared to 28.49 μg cm⁻² (8.62×10⁻⁸ μg cm⁻²) of furosemide, suggesting that drug delivery to the basal layers is a reality. The observation that a greater mass/area of furosemide permeated may be associated with the large SEM indicating that these results lacked reproducibility between samples. If integrity of the skin had decreased as furosemide is smaller than digoxin it is possible that it would penetrate the skin more effectively. It is also less lipophilic and therefore less likely to become trapped in a compartment of the skin. A larger percentage of loading of furosemide permeated the skin than digoxin, which was the same for the patches.

The ratio of moles that permeated the skin was D:F 1:8, supporting suggestions that furosemide permeated the skin more easily.

### Comparison Between Patches and Collodion

It was not possible to statically compare the patch formulation to the Collodion formulation, as although the rational behind the choice of ratio was the same, the actual ratios chosen for each formulation were slightly different. The discussion so far has compared the data obtained from the patches and Collodion, this next part of the discussion compares qualitative difference between the formulations.

### Vehicle Differences

A large amount of ethanol was present in the Collodion on application to the skin, comparatively there was no ethanol present in the patches. The ethanol in the Collodion formulation could be a potential problem in the treatment of genital warts. It may cause stinging as the nature of the wart tissue differs from cutaneous warts. It is also difficult to limit the application to the area of the wart without applying it to the surrounding sensitive mucus membranes. There are possible formulation solutions to overcome this, for example the inclusion of a local anaesthetic such as lignocaine to the formulation. However this would increase the number of actives in the formulation and could complicate the licensing of the product. Still, a degree of stinging may be acceptable to the patient bearing in mind the location of these warts and depending on the severity. On the other hand the inclusion of ethanol might aid percutaneous absorption to the basal cells. Dehydration of the keratinised skin may cause it to crack and forming microscopic pathways to the site of action. Ethanol is also known to act as a permeation enhancer by solubilising the lipids in regular skin. The extent of this in skin infected with the HPV is unknown, but perhaps will be reduced due to a lower proportion of lipids in this type of tissue.

Although the patches are impractical in the treatment of genital warts, their solids physical state means that limiting the application of the active to the healthy surrounding tissue, of cutaneous and plantar warts would not be difficult.

### Properties of the Dosage Form

The patch offers a thicker film than the Collodion, meaning that a larger mass of binary drug combination can be incorporated into the formulation, and perhaps offer a prolonged duration of treatment, increasing compliance. Thickness of film of Collodion is approximately 5-20 μcl limiting the amount of actives applied to the skin (Schaefer and Redelmirer, 1996) compared to approximately 1 mm of the patches. This suggests that movement of molecules from the upper surface of the patch through the bulk matrix to a greater extent in the patches, reducing frequency of dosing and aiding compliance. Both dosage forms are flexible, although there is little mobility in the wart tissue, flexible properties are required as only plantar warts are flat. The suitability of these patches in the treatment of common warts will be established in forthcoming clinical trials. Overall the formulation determines the kinetics and extent of percutaneous absorption, which has an impact upon the onset of action, duration and extent of a biological response.

### EXAMPLE 37

### Early Results of Patients with Plantar Warts Treated with Drug-in-Glue Dressing

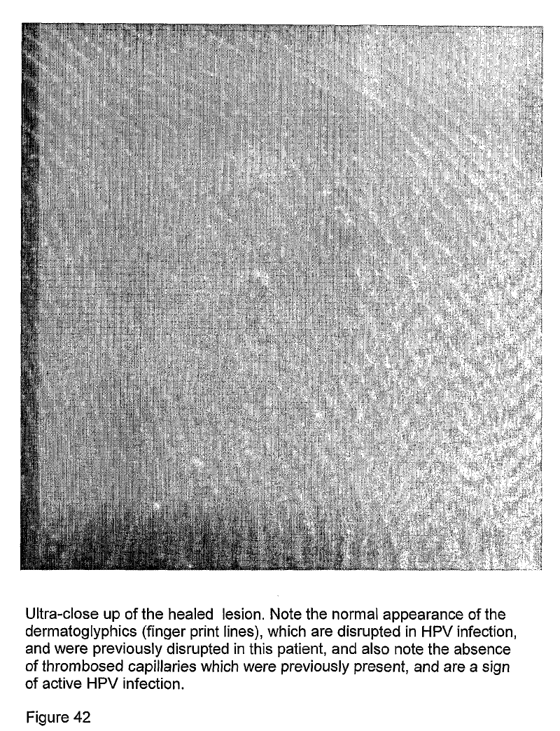

Patient PW 1

| | |
|---|---|
| Age | 43 |
| Sex | Male |
| Occupation | Self Employed |
| Lesion description | Highly keratinised lesion over the weight bearing |
| | aspect of the hallux right foot |
| HPV DNA | Results awaited |
| Duration of warts | Over 4 years |
| Previous Treatment | Tried chemical ablation with no effect, other |
| | destructive methods tried with no benefit |
| Formulation used | Drug-in-glue formulation Example 9 |
| Adverse effects | Nil |
| Systemic digoxin | Below limits of detection on three occasions |
| Blood Pressure | No significant change |
| Serum Potassium | Normal throughout |
| Duration of treatment | 21 days |
| Result of treatment | 4scopically at three weeks (see FIG. 42). |
| | Follow up continues on this patient |

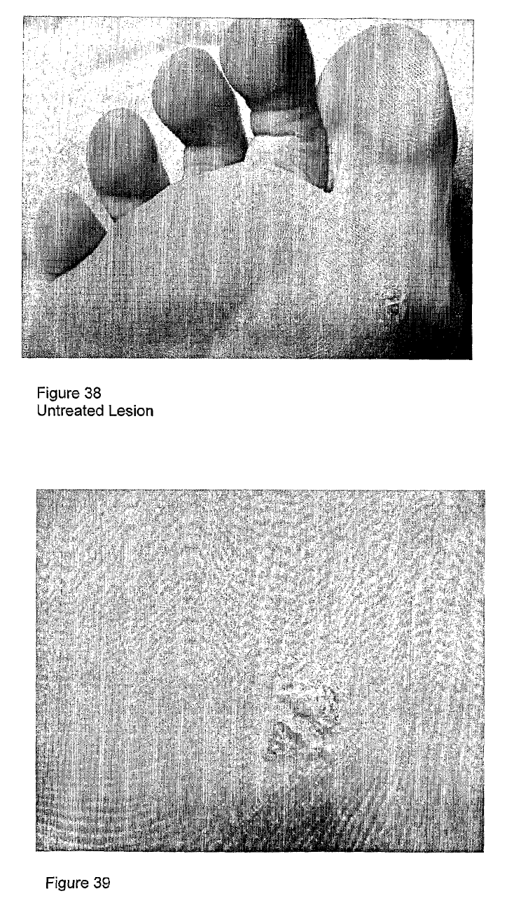

FIG. 38 shows the unrelated lesion on the underside of the patient's foot;

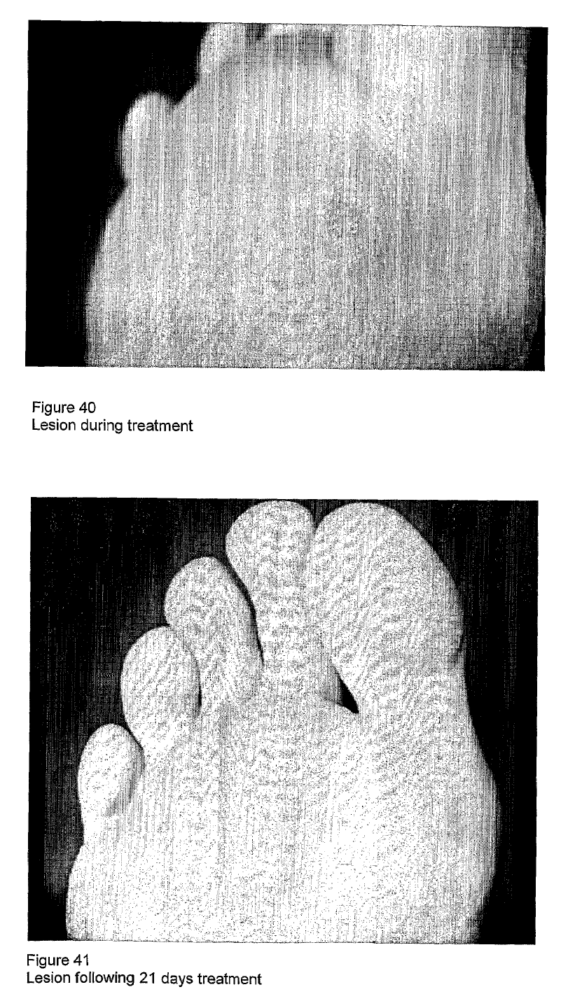

FIG. 39 is a closer view of the lesion in FIG. 40;

FIG. 40 shows the lesion during treatment with delivery means according to the invention;

FIG. 41 shows the lesion after 21 days treatment; and

FIG. 42 shows the healed lesion in ultra-close up.

In addition to the above described examples, the following additional embodiments demonstrate the in vitro release and permeation of Digoxin and Furosemide from transdermal delivery devices. Several drug-in-glue formulations containing differing amounts of Digoxin and Furosemide were compared for their rates of drug release, rates of drug permeation through porcine skin and the concentration of drug within the skin sample. The ratios of the active principles were varied to investigate optimum formulations for delivery of Furosemide and Digoxin to provide dermal saturation.

### Materials

Digoxin and Furosemide were purchased from Sigma, UK. Glue 1 was sourced from National Starch and Chemical Company. Al solvents and chemicals used for the release and permeability studies were purchased from Sigma. The porcine ear skin that was used as a skin barrier was purchased from a local abattoir.

### Test Protocol:

A convenient drug loading is 25 mg/mL of both Digoxin and Furosemide within the acrylate glue at a 1:1 ratio. If the total concentration of drug is maintained at 50 mg/mL then the following systems can be examined:

50 mg/mL Digoxin
46.7 mg/mL Digoxin and 3.3 mg/mL Furosemide (14:1 ratio)
40 mg/mL Digoxin and 10 mg/mL Furosemide (4:1 ratio)
30 mg/mL Digoxin and 20 mg/mL Furosemide (3:2 ratio)
25 mg/mL Digoxin and 25 mg/mL Furosemide (1:1 ratio)
20 mg/mL Digoxin and 30 mg/mL Furosemide (2:3 ratio)
10 mg/mL Digoxin and 40 mg/mL Furosemide (1:5 ratio)
3.3 mg/mL Digoxin and 46.7 mg/mL Furosemide (1:14 ratio)
50 mg/mL Furosemide
Plus a control using the glue only

The above systems measure ratios in a mass by mass form. Molar ratios of drugs were also examined at a 1:1 ratio of F:D, a 1:25 and a 1:100 ratio, and results provided in Table 13.

TABLE 13

| | Mass of drug per mL glue sample | |
|---|---|---|
| Molar ratio | with total drug at 50 mg/mL | |
| Furosemide:digoxin | Mass of furosemide (mg) | Mass of digoxin (mg) |
| 1:1 | 14.885 | 35.115 |
| 1:25 | 0.84 | 49.16 |
| 1:100 | 0.21 | 49.79 |

### Methods

### Drug Release Studies

Drug release from the patches into a solution of mobile phase was measured for the nine mass-ratio formulations. This was done to compare how the drug loading affects drug release.

### Drug Permeation Studies

Drug permeation through porcine ear skin was measured using Franz Diffusion cells where the amount of both drugs that permeated the tissue was measured over time and compared to the initial drug loading within the patch. The molar-ratio patches were used in this study. Pig's ear skin was used as a model membrane and the drug release through this tissue was measured using Franz cell apparatus. The skin was mounted above the receptor fluid that contains water:methanol:acetonitrile (40:30:30) as used for the mobile phase within the HPLC analysis.

The entire system was sealed to avoid moisture loss and samples were taken from the receptor fluid at intervals of 0, 4, 8, 12, 24, 48 and 72 hours. The receptor fluid was stirred continuously to ensure a homogenous receptor solution. The concentrations of both furosemide and digoxin within this fluid were measured via HPLC analysis. After 72 hours the skin was homogenised and the concentration of both drugs within this tissue was determined (via extraction) to note the “saturation” levels.

### Skin Saturation Studies

It has been well documented that skin has a capacity for the retention of drugs. It is generally thought that drugs with a higher log P value are retained to a greater extent within the skin. The amount of drug that was present in the skin sample at the end of the 72 hour period was measured via homogenisation of the skin onto which the patch had been administered and extraction of the drug. Each Franz cell was loaded with a patch of 2 cm diameter that would Q contain.

### Results

The cumulative amount of drug that is released from the glue or has penetrated the skin, Q (μg/cm²) was plotted against time in FIG. 45. The linear portion of such a slope (at least 5 data points used) was taken as being the steady state flux, Jss. The permeability coefficient, Kp (units=cm per time), the constant for each drug that determines how fast it is able to diffuse either through the glue to allow release or through the skin was then calculated as:

Kp=Jss/Cv

Where Cv is the concentration of the penetrant in the donor compartment (concentration of digoxin or furosemide within the patch, units=μg/cm³)

### Drug Release Studies:

Patches were made of the initial nine formulations and the drug release from these formulations into a solution of the mobile phase was measured.

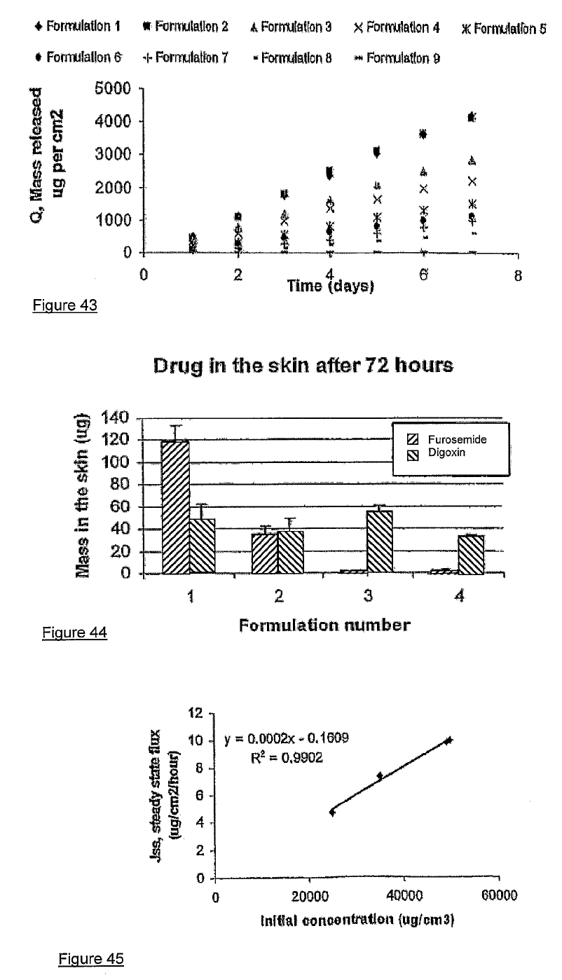

Some example data is shown below, the mass of digoxin released from each formulation was plotted against time in FIG. 43. A similar plot was constructed for furosemide.

The gradient of these results was calculated and is a measure of the steady state flux from the patches, Jss. Division of the steady state flux by the initial concentration gives the permeability coefficient, this value is a constant that determines the rate of drug release from the patch. The table below provides the data that measures both the amount of drug release from each patch at 4 days, the steady state flux and the permeation coefficient for each formulation.

The rates of both digoxin and furosemide release from the patches are listed in the table below.

TABLE 14

| | Initial drug | | Mass | | Steady | | Permeation | |
|---|---|---|---|---|---|---|---|---|
| | concentration | | released at 4 | | state flux, Jss | | coefficient, Kp | |
| | (ug/cm3) | | day (ug) | | (ug/cm2/day) | | (cm/sec) | |
| Formulation | Digoxin | Furosemide | Digoxin | Furosemide | Digoxin | Furosemide | Digoxin | Furosemide |
| 1 | 50000 | 0 | 2390.89 | 0.00 | 621.37 | 0 | 1.44E−07 | |
| 2 | 46700 | 330 | 2507.63 | 960.93 | 628.95 | 241.45 | 1.56E−07 | 8.47E−07 |
| 3 | 40000 | 10000 | 1637.95 | 2092.43 | 424.26 | 535.29 | 1.23E−07 | 6.20E−07 |
| 4 | 30000 | 20000 | 1365.44 | 4539.82 | 333.04 | 1181.9 | 1.28E−07 | 6.84E−07 |
| 5 | 25000 | 25000 | 821.30 | 4323.66 | 235.74 | 1322 | 1.09E−07 | 6.12E−07 |
| 6 | 20000 | 30000 | 635.54 | 5536.11 | 173.14 | 1574.3 | 1.00E−07 | 6.07E−07 |
| 7 | 10000 | 40000 | 403.23 | 6814.75 | 155.79 | 2086.4 | 1.80E−07 | 6.04E−07 |
| 8 | 3300 | 46700 | 236.42 | 8987.90 | 96.10 | 2595.1 | 3.37E−07 | 6.43E−07 |
| 9 | 0 | 50000 | 0.00 | 8771.84 | 0.00 | 2612.6 | | 6.05E−07 |
| | | | | | | Kp mean | 1.60E−07 | 0.53E−07 |
| | | | | | | Kp stdev | 7.62E−08 | 8.30E−08 |

Table 14 shows that at similar concentration values, furosemide is released to a greater extent than digoxin, e.g. compare formulations 1 and 9. The steady state flux for each drug increases as the initial loading of drug within the patch increases. This is as expected as the drug is released from the patch due to a concentration gradient that exists between the drug loading and release medium. The permeation coefficient is a measure of the rate of drug release in cm per second of each drug from the patch. These values are relatively constant for all formulations which indicates that the two drugs do not interfere in the release of one another. The Kp values for each drug alone are similar to the values in patches that contain both drugs. Kp for furosemide is approximately four times greater than Kp for digoxin, this is likely to be due to the comparatively smaller size of furosemide.

The table below shows the data for the drug released from the patches that has penetrated the skin.

TABLE 15

| | Initial drug | | Mass | | Steady | | Permeation | |
|---|---|---|---|---|---|---|---|---|
| | concentration | | released at 48 | | state flux, Jss | | coefficient, Kp | |
| | (ug/cm3) | | house (ug) | | (ug/cm2/hour) | | (cm/sec) | |
| Formulation | Digoxin | Furosemide | Digoxin | Furosemide | Digoxin | Furosemide | Digoxin | Furosemide |
| 1 | 25000 | 25000 | 269.43 | 1330.63 | 4.7184 | 31.966 | 5.24E−08 | 3.55E−07 |
| 2 | 35115 | 14885 | 444.07 | 1162.70 | 7.33 | 25.25 | 5.80E−08 | 4.71E−07 |
| 3 | 49169 | 840 | 447.30 | 32.82 | 9.742 | 0.7051 | 5.50E−08 | 2.33E−07 |
| 4 | 49790 | 210 | 469.23 | 19.64 | 9.9154 | 0.5045 | 5.53E−08 | 6.67E−07 |
| | | | | | | Mean | 5.52E−08 | 4.32E−07 |
| | | | | | | stdev | 2.27E−09 | 1.85E−07 |

Table 15 shows the penetration of the skin, both the flux values and permeation coefficient values are much lower than the release of the drug from the formulations listed in the table above. This is expected and reflects the barrier properties of the skin. Furosemide penetrates the skin to a greater extent than digoxin as demonstrated by the permeation coefficient which is nearly eight times higher than digoxin.

The drug that accumulated in the skin was also measured. The drug that was present in a 2 cm diameter cross section of skin was calculated for all four formulations.

The level of digoxin appeared to be independent of the loading formulation, indicating that the skin was saturated with digoxin at a concentration of 40 ug over 3.14 cm² or 12.73 μg/cm². Furosemide did not accumulate within the skin and permeated directly through the skin. The concentration measured at 72 hours was a transient indication of furosemide within the skin that was dependant upon the loading concentration. Results are shown in FIG. 44.

The rate of furosemide release from the patch, Kp for the patch was 6.53×10⁻¹⁰ cm per second, this was not greatly faster than the rate of furosemide penetrating porcine ear skin at 4.32×10⁻⁸ cm/second.

Digoxin was considerably slower both in terms of drug release and also in terms of skin penetration with permeation coefficients of 1.60×10⁻⁷ cm/s and 5.52×10⁻⁸ cm/s for the patch and skin respectively.

If the initial patch concentration for digoxin is plotted against the steady state flux rate through the skin, as shown in FIG. 45, it can be seen that for the flux to be greater than zero the initial concentration within the patch must be 804.5 μg/cm³.

25 000 μg/cm3 was the lowest concentration used within the skin study. The required flux for effective therapy was 25 μg per day, if this is assumed to come from a patch with a surface area of 1 cm² then the loading dose should be:

Flux=25 jig per day per cm²=1.04 μg per cm² per hour, thus a loading dose of 6004.5 μg per cm³ is required.

However, this study enhanced the overall penetration of digoxin through the skin as a very lipophilic substance was used in the donor phase to enhance the concentration gradient to maximise skin penetration of both digoxin and furosemide.

Two particularly effective drugs are Digoxin and Furosemide and examples of their 50% plaque Inhibitory Concentrations (IC50) are given below (Table A). The IC50 is an often quoted index of antiviral drug potency useful and convenient when comparing different drugs. Used separately, both Digoxin and Furosemide clearly inhibit the replication of a broad range of viruses.

TABLE A

| | | Digixin IC50 | Furosemide IC50 |
|---|---|---|---|
| Virus | Host Cell | (ng/ml) | (ug/ml) |
| Adenovirus | A549 | 15 | 300 |
| Cytomegalovirus | MRC5 | 20 | 600 |
| Varicella-Zoster virus | MRC5 | 50 | 500 |
| Herpes simplex virus | MRC5 | 25 | 600 |
| Herpes simplex virus | BHK21 | 30 | 800 |
| Herpes simplex virus | Vero | 60 | 1000 |

An alternative index of antiviral activity, however, demonstrates the true potency of these drugs. Since ICVT permits the synthesis of non infectious virus proteins and those proteins cause, in part, the changes in cell pathology (cytopathic effect) that form the basis of IC50 determinations, the potency of these drugs is underestimated by IC50 determinations. An alternative index measures instead the total number of infectious virus particles produced by infected cells.

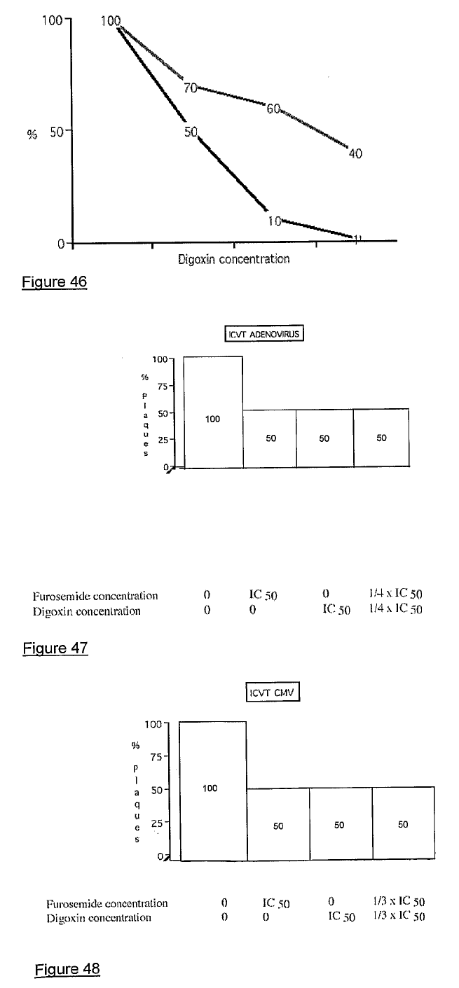

Using Digoxin, for example, inhibition of Herpes Simplex Virus plaque production of between 40% and 60% ie the IC50 effect (upper line on graph; FIG. 46) corresponds to between 90% and 99% inhibition of infectious virus particle production (lower line on graph; FIG. 46).

Using Digoxin and Furosemide individually, each at their IC50, against another virus, namely feline herpesvirus, virus replication is almost completely inhibited (Table B). While the production of infectious virus is reduced by 98.5% (Digoxin) and 99.5% (Furosemide) there remains a low level of virus replication; i.e., 1.5% (Furosemide) and 0.5% (Digoxin).

TABLE B

| | Virus particles | Virus particles | |
|---|---|---|---|
| | per cell | per cell | Virus particles per cell |
| Virus | No Drug | Digoxin IC50 | Furosemide IC50 |
| Feline herpes | 50 | 0.75 | 0.25 |
| virus | | | |

It is possible, however, to effectively eliminate this residual, low level of virus replication by using the drugs in combination. The combined antiviral effect being greater than when the drugs are applied separately; the drugs are synergistic (Table C).

TABLE C

| | Virus particles per cell | Virus particles per cell Digoxin |
|---|---|---|
| Virus | No Drug | IC50 and Furosemide IC50 |
| Feline herpes | 50 | 0.00001 |
| virus | | |

Thus, virus replication is reduced by 99.99999%.

The replication of other viruses is also most effectively inhibited by using the drugs in combination, for example, Varicella Zoster Virus (VZV). It is impossible, however, to quantify the precise number of infectious VZV particles involved since VZV is a highly cell-associated virus. Instead the effects of individual and combined IC50s on virus plaque formation are compared (Table D).

Furosemide and Digoxin, each at their respective IC50s inhibited VZV plaque formation, as expected by about 50%; Furosemide 33/61 plaques and Digoxin 21/61 plaques. However, when both drugs at their IC50s were applied in combination. VZV plaque formation was completely inhibited at the low multiplicity of infection (Low MOI). Indeed, VZV plaque formation was completely inhibited when there was one hundred-fold more infection virus in the test system; the High MOI. Using this index of potency, the drugs were, more than one hundred-fold more potent when applied in combination.

TABLE D

| | High MOI¹ | Intermediate MOI² | Low MOI³ |
|---|---|---|---|
| Control | TNTC | TNTC | 61 |
| Furosemide IC50 | TNTC | TNTC | 33⁴ |
| Digoxin IC50 | TNTC | TNTC | 21⁵ |
| Furosemide IC50 | 0⁸ | 0⁷ | 0⁶ |
| And Digoxin IC50 | | | |

¹100 × Low Multiplicity Of Infection
²10 × Low Multiplicity Of Infection
³Low Multiplicity Of Infection
⁴50% plaque inhibition
⁵50% plaque inhibition
⁶100% plaque inhibition
⁷100% plaque inhibition
⁸100% plaque inhibition

Comparison of the combined effects of fractional IC50s provides another index by which to compare the relative potencies the two drugs alone and in combination. In the example below, using Adenovirus, only one quarter of the IC50 of each drug is sufficient, when used in combination, to elicit the same antiviral effect as the IC50 of either drug alone (FIG. 47).

The same phenomenon maintains with Cytomegalovirus (CMV), another strongly cell-associated virus; when the two drugs are used in combination, only one third of the IC50 of each drug is sufficient to elicit the same antiviral effect as the IC50 of either drug alone (FIG. 48).

In summary, Digoxin and Furosemide are synergistic when applied to ICVT. Due to the unique mechanism of antiviral activity (ICVT), the standard IC50 index undervalues true drug potency although the increased, combined effect remains clear using this index.

Most strikingly, the production of infectious virus is decreased by 99.99999% when the drugs are used in combination.

### The Comparative Solubilities and ICVT-Potencies of Digoxin, Digitoxin and Lanoxin (IV)

### 1) Comparative ‘ICVT-ivities’ (Ionic Contra-Viral Therapy-Activities)

Solutions of Digoxin and Digitoxin were prepared from powder to a concentration of 250 ug per ml in 70% ethanol and their ICVT-ivities compared with the ‘standard’ Digoxin preparation; i.e. IV Lanoxin, which is supplied at 250 ug per ml in 10% ethanol.

The ID₅₀ values of Digoxin prepared from powder and Lanoxin (circles) (FIG. 49) were very similar, i.e. 60 ng per ml. Digitoxin (squares) appeared to be marginally better with an ID50 of 30 ng per ml.

### 2) Comparative Solubilities

Saturated solutions of Digoxin and Digitoxin (were prepared in 90% ethanol and their ‘ICVT-ivities’ compared with the ‘standard’ Digoxin preparation; i.e. Lanoxin.

Digoxin solution prepared from powder was as effective as Lanoxin (circles) (FIG. 50).

Digitoxin (squares) was again more effective than Digoxin.

Digitoxin is more soluble than Digoxin; preparation of a saturated solution (17.5 mg per ml) in 90% ethanol will enable use at a maximum concentration of 486 ug per ml in a ‘safe-ocular-concentration (2.5%) of ethanol.

Digoxin was previously used at a concentration of 62.5 ug per ml.

486 ug per ml is approximately eight times more concentrated and if Digitoxin is indeed twice as potent then it might be possible to use what would effectively be 16× the previous ‘dose’. Toxicity at this higher concentration will, of course, need to be examined.

### 3) Comparative ‘ICVT-ivities’

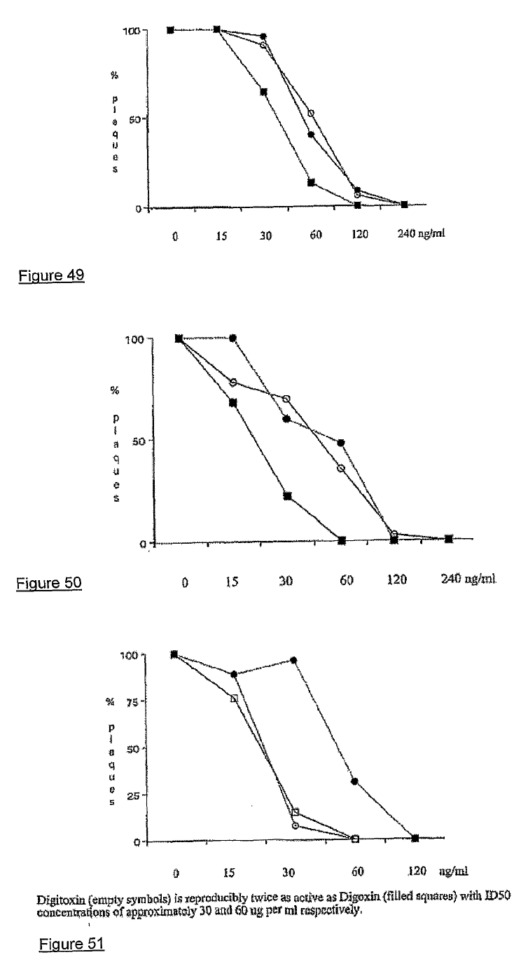

Fresh solutions of Digoxin and Digitoxin were prepared from powder to a concentration of 250 ug per ml in 70% ethanol and their ICVT-ivities again compared with the ‘standard’ Digoxin preparation; i.e. IV Lanoxin in order to further examine their relative potencies. Results are depicted in FIG. 51.

In addition to the above examples, the following further embodiments demonstrate the effects of Furosemide and Digoxin, individually and in combination, on Varicella Zoster virus replication in vitro and an MRC5 cell replication and metabolism.

### 1.1. MRC5

MRC5 cells (Jacobs et al 1970), a line derived from human embryonic lung tissue, were obtained from BioWhittaker. Cells were propagated in Eagles medium (Life Technologies Ltd) supplemented with 10% (v/v) foetal calf serum (Life Technologies Ltd). MRC5 cells were used for Varicella Zoster Virus (VZV) stock production and in experiments investigating the effects of Ionic Contra-Virals on VZV replication.

### 1.2. Cell Morphology

The maximum drug concentration permitting normal cell was determined by incubation of sub-confluent cultures in drug-containing media for 72 hours. Cells were examined directly using phase contrast microscopy.

### 1.3. Cell Replication

The maximum drug concentration permitting cell replication was determined similarly; after 72 hours cells were harvested and counted. A tenfold increase in cell number was taken to be representative of normal cell replication (minimally three population doublings in 72 hours).

### 1.4. MTT (Dimethylthiazol Diphenyltetrtazolium Bromide) Assay

MTT assays were performed as described in Antiviral Methods and Protocols (Kinchington, 2000).

### 1.5. Varicella Zoster Virus (VZV)

The Ellen strain of VZV was obtained from the American Type Culture Collection.

### 1.6. VZV Monolayer Plaque Inhibition Assay

VZV infected cells were assayed on preformed monolayers of MRC5 cells in 5 cm petri dishes by inoculation with 5 ml of infected cell suspension and incubation for 72 hours, or until viral cpe was optimal. Cells were fixed with formol saline and stained with carbol fuchsin.

### 2. Results

### 2.1. The Effect of Furosemide on VZV Replication In Vitro.

Furosemide at a concentration of 1.0 mg/ml was very well tolerated by MRC5 cells; there was no adverse effect on cell morphology and cells replicated. Furosemide inhibited VZV plaque formation by 50% at this concentration.

Furosemide ID 50; 1.0 mg/ml. [Table E]

VZV replication was completely inhibited by Furosemide at a concentration of 2.0 mg/ml.

### 2.2 The Effect of Digoxin on VZV Replication In Vitro

Digoxin at a concentration of 0.05 ug/ml was very well tolerated by MRC5 cells; there was no adverse effect on cell morphology and cells replicated. Digoxin inhibited VZV plaque formation by 50% at this concentration.

Digoxin ID 50; 0.05 ug/ml. [Table E]

VZV replication was completely inhibited by Digoxin at a concentration of 0.1 ug/ml.

### 2.3. The Effects of Furosemide and Digoxin on VZV Replication In Vitro

VZV replication was completely inhibited by Furosemide and Digoxin in combination at their individual ID 50 concentrations [Table E]. The combined dosage was equally well tolerated by MRC5 cells; there was no adverse effect on cell morphology and cells replicated.

### The Effects of Furosemide and Digoxin, Individually and in Combination, on Varicella Zoster Virus Replication In Vitro [Table E]

NB. There was a ten-fold difference between adjacent multiplicities if infection (MOI)

TABLE E

| | HIGH | INTERMEDIATE | LOW |
|---|---|---|---|
| | MOI | MOI | MOI |
| CONTROL | TNTC* | TNTC | 61 |
| Furosemide 0.5 mg/ml | TNTC | TNTC | 33¹ |
| Furosemide 1.0 mg/ml | TNTC | TNTC | 16 |
| Furosemide 2.0 mg/ml | 0² | 0² | 0² |
| Digoxin 0.025 ug/ml | TNTC | TNTC | 55 |
| Digoxin 0.050 ug/ml | TNTC | TNTC | 21³ |
| Digoxin 0.100 ug/ml | 0⁴ | 0⁴ | 0⁴ |
| Furosemide 0.5 ug/ml | 0⁵ | 0⁵ | 0⁵ |
| Digoxin 0.050 ug/ml | | | |

TNTC* Too numerous to count.
¹Furosemide 50% Plaque Inhibitory Dose [ID 50] 0.5 mg/ml.
²Furosemide completely inhibited VZV at a concentration of 2.0 mg/ml.
³Digoxin 50% Plaque Inhibitory Dose ID 50; 0.05 ug/ml.
⁴Digoxin completely inhibited VZV replication at a concentration of 0.1 ug/ml.
⁵VZV replication was completely inhibited by Furosemide and Digoxin in combination at their individual ID 50 concentrations.

### 2.4. The Effect of Furosemide on MRC5 Cell Replication

Uninfected MRC5 cells replicated to normal yields in the presence of Furosemide at a concentration of 1.0 mg/ml, the same concentration as the VZV ID50.

### 2.5. The Effect of Digoxin on MRC5 Cell Replication

Uninfected MRC5 cells replicated to normal yields in the presence of Digoxin at a concentration of 0.05 ug/ml, the same concentration as the VZV ID50.

### 2.6. The Effects of Furosemide and Digoxin on MRC5 Cell Replication

Uninfected MRC5 cells replicated, though not to normal yields, in the presence of both Furosemide and Digoxin at their VZV ID50 concentrations. At these concentrations, VZV replication was completely inhibited.

### 2.7. The Effects of Furosemide and Digoxin on MRC5 Cell Metabolism

The effects of Furosemide and Digoxin on MRC5 cell metabolism were measured using the MTT assay. There were normal levels of metabolism in uninfected cells incubated with either Furosemide or Digoxin at their VZV ID 50 concentrations. There was normal metabolism in uninfected cells incubated with both Furosemide and Digoxin at their VZV ID 50 concentrations. In combination at these concentrations VZV replication was completely inhibited (2.3).

In addition to the above examples, the following further embodiments demonstrate the efficacies of alternative diuretics and cardiac glycosides.

Examples of Thiazide (Hydrochlorothiazide and Metolazone), Sulphonylurea (Tolbutamide), Sulphonamide (Furosemide, Acetazolamide, Bumetanide, Torasemide and Ethacrynic acid) and K sparing diuretic (Amiloride) were tested for ICVT activity. The cardiac glycosides Digoxin, Digitoxin, Lanoxin and Strophanthin G were also tested.

Using Herpes simplex virus (HSV), 50% plaque inhibitory dose (1D50) were established using the standard plaque inhibition assay. Various solvents were required to facilitate testing and these were sometimes detrimental to tissue culture, depending upon their concentration. Certain compounds elicited potent ICVT activity (Furosemide, Digoxin, Lanoxin and Digitoxin) and these were active at high dilution; experimental conditions in which solvent toxicity was excluded.

Other compounds elicited only ‘borderline’ CVI activity. These compounds (Acetazolamide, Tolbutamide and Hydrochiorthiazide) were further tested using alternative solvents in the same test system (ie the plaque inhibition assay) and others (Bumetanide, Torasemide, Tolbutamide and Hydrochlothiazide) in a more sensitive test for ICVT activity in which the effects on virus yields were determined. The effects of cardiac glycosides Digoxin and Strophanthin on virus yields were also tested in this assay.

| | |
|---|---|
| Thiazide | |
| Hydrochiorothiazide | |
| Solvent: Ethanol 10% 5 mg/ml | |
| HSV Plaque 1D50 Negative @ 2.5 mg/ml | − |
| Solvent: NaOH 1% aqueous 10 mg/ml | |
| HSV Plaque 1D50 400 ug/ml Borderline | +/ |
| HSV yield reduced to zero at 600 ug/ml | + |
| Metolazone | |
| Solvent: PEG 10 mg/ml | − |
| Solvent: PG 0 mg/ml | − |
| Sulphonylurea | |
| Tolbutamide | |
| Solvent: NaOH 1% aqueous 10 mg/ml | |
| HSV Plaque ID50 500 μg/ml Borderline | +/ |
| Solvent: PEG 10 mg/ml | |
| HSV Plaque 1D50 500 μg/ml Borderline | +/ |
| HSV yield reduced to zero 300 μg/ml | + |
| Solvent: PG 10 mg/ml | |
| HSV Plaque ID50 500 μg/ml Borderline | +/− |
| HSV yield reduced to zero 300 μg/ml | + |
| Solvent IPA 10 mg/ml | |
| HSV Plaque 1D50 250 μg/ml Borderline | +/ |
| Sulphonamide | |
| Furosemide | + |
| Solvent: aqueous (IV) 10 mg/ml | |
| HSV Plaque 1D50 1 mg/ml | |
| Acetazolamide | |
| Sigma | |
| Solvent: PEG 40 mg/ml | |
| HSV Plaque 1D50 Negative @ 500 μg/ml | − |
| Solvent: PG 7 mg.ml | |
| HSV Plaque 1D50 Negative @ 100 μg/ml | − |
| Bumetanide | |
| Solvent: (IV) Aqueous 500 μg/ml | |
| HSV Plaque 1D50 Negative @ 100 μg/ml | − |
| HSV yield reduced Borderline | +/− |
| Torasemide | |
| Qemaco | |
| Solvent: NaOH 1% aqueous 5 mg/ml | |
| HSV Plaque 1D50 60 μg/ml Borderline | +/ |
| HSV yield unaffected at 90 μg/ml | − |
| Ethacrynic acid | |
| Solvent; (IV) Aqueous 100 μg/ml | |
| HSV Plaque 1D50 25 μg/ml Negative | |
| K sparing diuretic | |
| Amiloride | |
| Solvent: Aqueous 500 μg/ml | |
| HSV Plaque ID5O 250 μg/ml | +/− |
| Cardiac glycoside | |
| Digoxin (IV) 250 μg/ml | |
| HSV Plaque 1D50 60 ng/ml | + |
| HSV yield reduced | + |
| Digitoxin | |
| Solvent: Ethanol | |
| HSV Plaque ID50 30 ng/ml | + |
| HSV yield reduced | + |
| Lanoxin (IV) 250 μg/ml | |
| HSV Plaque ID5O 60 ng/ml | + |
| HSV yield reduced | + |
| Strophanthin G | |
| Solvent: Aqueous | |
| HSV Plaque 1D50 1 mg/ml Cytotoxic | |
| HSV yield reduced Borderline | +/− |

Thus, these and other loop diuretics and/or cardiac glycosides will have utility in transdermal active principle delivery means, especially when provided in or with an adhesive.

1-32. (canceled)

33. Transdermal active principle delivery means comprising a skin adherent or otherwise skin-tolerant substrate applicable to a skin area affected by DNA virus, which substrate includes a composition for treating DNA viral infections comprising a transdermally effective carrier medium including at least one active principle selected from the group consisting of diuretic agents (e.g. loop diuretic agents) and/or cardiac glycoside agents.

34. Delivery means as claimed in claim 33, comprising one or more loop diuretic agents in conjunction with one or more cardiac glycoside agents.

35. Delivery means as claimed in claim 33, wherein the diuretic is one or more of the following: Furosemide, bumetranide, ethacrynic acid and torazemide.

36. Delivery means as claimed in claim 35, wherein the diuretic is furosemide.

37. Delivery means as claimed in claim 33, wherein the cardiac glycoside is a digitalis glycoside comprising one or more of the following: digoxin, digitoxin, medigoxin, lanatoside C, proscillaridin, k strophantin, peruvoside and ouabain.

38. Delivery means as claimed in claim 37, wherein the cardiac glycoside is digoxin.

39. Delivery means as claimed in claim 33, wherein the carrier medium comprises a pharmaceutically acceptable active principle-in-adhesive formulation.

40. Delivery means as claimed in claim 39, wherein the adhesive comprises acrylic polymer adhesive, preferably dissolved or dispersed within an alkyl ester solvent, for example, ethyl acetate.

41. Delivery means as claimed in claim 33, wherein the carrier medium comprises one or more pharmaceutically acceptable excipients to aid release and/or penetration of the active principle(s).

42. Delivery means as claimed in claim 33, wherein the carrier medium comprises one or more dermally acceptable solvents.

43. Delivery means as claimed in claim 42, wherein the solvent comprises one or more of the following: a monohydric alcohol, e.g. methanol, ethanol, propanol, an alkyl ester, e.g. ethyl acetate, an alkylene glycol, e.g. propylene glycol and water.

44. Delivery means as claimed in claim 33, wherein the carrier medium further includes at least one viscosity modifier such as carbopol or hydroxypropyl cellulose.

45. Delivery means as claimed in claim 33, wherein the rate of release of the active principle(s) from the composition is greater than 10 μg/cm²/24 hrs, preferably greater than 20 μg/cm²/24 hrs, more preferably greater than 50 μg/cm²/24 hrs, most preferably greater than 100 μg/cm²/24 hrs.

46. Delivery means as claimed in claim 33, wherein the active principle loading upon or within the substrate is greater than 0.5 mg/cm², preferably greater than 1.0 mg/cm², more preferably greater than 1.5 mg/cm² most preferably greater than 2.0 mg/cm² of active principle(s) per square centimetre of that part of the delivery means capable of delivering the principle(s) to the skin from the composition.

47. Delivery means as claimed in claim 34, wherein the molar ratio of diuretic to cardiac glycoside is in the range of 100 to 0.1 moles of glycoside:mole of diuretic.

48. Delivery means as claimed in claim 39, wherein the weight ratio of active principle(s):adhesive formulation is in the range of 1:5-20, preferably 1:5-15 more preferably 1:8-12.

49. Delivery means as claimed in claim 33, wherein a skin adherent substrate is used wherein a reservoir containing the composition is affixed to the substrate and a releasable layer affixed to the reservoir.

50. Delivery means as claimed in claim 49, in the form of an adhesive patch comprising an island reservoir impregnated with the composition.

51. Delivery means as claimed in claim 33, wherein a skin tolerant adherent membrane is used comprising a lacquer composition.

52. Delivery means as claimed in claim 51, in which the lacquer is a flexible Collodion lacquer.

53. Delivery means as claimed in claim 52, wherein the collodion comprises a mixture containing benzoin tincture, paraffin wax and methylcellulose.

54. Delivery means as claimed in claim 53, wherein the collodion is diluted with an ether solvent.

55. Delivery means as claimed in claim 51, wherein the composition comprising the active principle(s) is applied and adhered directly to a surface of the dried lacquer in the absence of an absorbent reservoir.

56. Delivery means as claimed in claim 51, wherein the composition comprising active principle(s) includes at least one solvent in which the principle is (are) dissolved and/or dispersed.

57. Delivery means as claimed in claim 56, wherein the solvent comprises an alcohol with or without water.

58. Delivery means as claimed in claim 57, wherein the alcohol is a monohydric alcohol such as an alkanol, for example, ethanol.

59. Delivery means as claimed in claim 51, wherein a solvent is present in which the principle(s) is (are) dissolved and/or dispersed and wherein the ratio of principle:lacquer composition:solvent is in the range 0.01:1-10:1-10.

60. Delivery means as claimed in claim 33, wherein the composition for treating DNA virus is effective as a topical application against the effects of human papillomavirus (HPV) infection.

61. Delivery means as claimed in claim 33, in which the composition is effective as a topical application to warts such as plantar warts and/or hand/finger and/or genital warts.

62. A method of making delivery means, which method comprises formulating a composition for treating DNA viral infections comprising a transdermally effective carrier medium including at least one active principle selected from the group consisting of diuretic agents (e.g. loop diuretic agents) and/or cardiac glycoside agents, providing a flexible collodion lacquer and allowing this to set or otherwise become tacky, and applying the composition directly to the set or tacky Collodion lacquer and optionally applying a releasable protective layer to the exposed composition.

63. Use of a diuretic and/or a cardiac glycoside for the manufacture of a topical medicament for the treatment of DNA viral infections, for example human papillomavirus infection, wherein said topical medicament comprises a flexible collodion layer or adhesive.

64. A method of treating human papillomavirus infection in a subject, the method comprising applying a topical medicament to the subject, the topical medicament comprising a diuretic and/or cardiac glycoside and a flexible collodion layer or adhesive.
